(19) United States
(12) Patent Application Publication
(10) Pub. No.: US 2010/0021391 A1
(43) Pub. Date: Jan. 28, 2010

(54) NOVEL NANOPARTICLES FOR BIOFILM TARGETING

(75) Inventors: Trevor Douglas, Bozeman, MT (US); Mark J. Young, Bozeman, MT (US); Peter Suci, Bozeman, MT (US)

Correspondence Address: COOLEY GODWARD KRONISH LLP;ATTN: Patent Group, Suite 1100, 777 - 6th Street, NW, WASHINGTON, DC, 20001, US

(73) Assignee: MONTANA STATE UNIVERSITY, Bozeman, MT (US)

(21) Appl. No.: 11/777,944

(22) Filed: Jul. 13, 2007

Related U.S. Application Data
(60) Provisional application No. 60/830,830, filed on Jul. 14, 2006.
(60) Provisional application No. 60/830,838, filed on Jul. 14, 2006.

(51) Int. Cl. A61K 49/14; A61K 49/04; A61K 49/22

(52) U.S. Cl. 424/9.34; 424/9.4; 424/9.5

(57) ABSTRACT

The present invention is directed to novel compositions and methods utilizing nanoparticles comprising protein cages for delivery of imaging and antimicrobial agents to biofilm forming bacterial colonies.

### FIELD OF THE INVENTION

The present invention is directed to novel compositions and methods utilizing nanoparticles comprising protein cages for delivery of imaging and antimicrobial agents to biofilm forming bacterial colonies.

### BACKGROUND OF THE INVENTION

Protein cage structures have been used as carriers for a variety of different agents. A variety of cages, including viral protein based and non-viral protein based, and uses thereof can be found for example in U.S. Pat. Nos. 6,180,389 and 6,984,386, as well as U.S. patent application Ser. Nos. 10/358,089 filed Feb. 3, 2003, 10/441,962 filed May 19, 2003, 11/430,632 filed Apr. 27, 2006, 11/415,485 filed Apr. 27, 2006, 60/736,041 filed Nov. 9, 2005, and the U.S. patent application filed on Jul. 14, 2006 entitled “Novel Nanoparticles Containing Bacterial Protein Subunits and Uses Thereof,” each of which is incorporated herein by reference in its entirety, and in particular for the compositions and methods of making and methods of use described therein.

Biofilm infections in humans are highly structured, matrix-encased communities of microorganisms, which are not tractable to conventional treatment. Biofilms are responsible for as much as 80% of all human infections and as such are a significant threat to human health in both the civilian and military sectors. Microorganisms in biofilms express resistance (as much as 1000 times the ‘normal’ dose of antimicrobials is required). Conventional antimicrobials are not effective against biofilm based infections and are therefore not usually effective.

A universal characteristic of biofilm infections is that once they become established, they become chronically recalcitrant to both host defenses and antimicrobial therapies. Emerging and proposed treatments are directed at disrupting the biofilm phenotype. For the foreseeable future, the only reliable cure for chronic biofilm infections remains surgical removal of the infected tissue and/or associated implant. In terms of our current understanding, the most cost-effective approach for treatment of biofilm infections is early detection followed by standard dosing with an appropriate antimicrobial. Availability of a non-invasive means for sensitive detection and classification of biofilm infections would provide a critical tool for early diagnosis, enhance the efficacy of emerging treatment schemes, and contribute to the efficiency and precision of surgical procedures.

Despite significant progress in characterizing biofilm genetic programs [Sauer 2002; Whiteley 2001; Beenken 2004], community structure [Foster 2004], physiological heterogeneity [Werner 2004], architecture [Lequette 2005] and multicellular behavior [Davies 1998; De Keivit 2001] there appears to be no forthcoming remedy for treatment of biofilm infections just on the horizon. Current understanding indicates that the ability of biofilms to thrive despite challenges by both host defense mechanisms and antimicrobial agents originates from a complex interplay of community interactions that are in some cases regulated at the level of multicellular behavior [Costerton 1999; Stewart 2001; Lewis 2001; Davies 2003]. Schemes for treatment of biofilm infections generally involve application of an agent that will disrupt some aspect of biofilm community structure [Sakakibara 2002] or behavior [Anguige 2004]. Proposed treatments include development of agents that will inactivate biofilm subpopulations that are tolerant to antimicrobial agents as a result of genetic programming [Lewis 2001] and induction of biofilm detachment by introduction of signaling molecules [Davies 2003]. Practical application of any of these strategies will require diagnosis of the infection as a biofilm infection, identification of the pathogen, and methods for monitoring the success of the treatment program. Non-invasive imaging techniques will play a prominent role in this overall treatment process.

One relatively simple strategy for controlling biofilm infections may be early detection followed by appropriate treatment with a conventional antimicrobial agent. In vitro studies indicate that there is an early period in biofilm development when the biofilm is still vulnerable to eradication with clinically acceptable doses of conventional antimicrobial agents [Anwar 1989; Anwar 1990; Anwar 1992 a, b; Kumon 1995; Williams 1997; Amorena 1999; Aaron 2002]. Although this window of vulnerability is relatively short (hours to days) in in vitro studies, biofilms develop more slowly under less benign in vivo conditions on implants in animal models [Ward 1992; Gracia 1998; Monzon 2002] indicating that this period of vulnerability is lengthened considerably in vivo. Non-invasive imaging techniques provide an ideal tool for implementing this preventative strategy.

Contrast enhanced MRI offers outstanding potential as a technique for sensitive detection of biofilm infections. The quality of MR images of soft tissues is superior to those produced by any other technique, and involves no exposure of the patient to potentially harmful radiation. MRI data can be used to assess the physiological state of tissues. Thus, state-of-the art MRI combines capabilities of both CT and nuclear medicine [Wolfbarst 1999]. The invisibility of bone is considered to enhance image quality and information content [Oldendorf 1987]. The primary disadvantage of MRI is that it cannot be used in cases involving pacemakers. Use of MRI has been advocated for diagnosis of vascular graft infections [Spartera 1997; Williamson 1989] and to determine the extent of infection in cases of osteomyelitis when diagnosis is uncertain [Mader 1996]. Due to its combination of sensitivity and specificity MRI has become a method of choice for diagnosis of vertebral infection [Jevtic 2004] and osteomyelitis of the diabetic foot [Lipsky 2004]. MRI is primarily limited it its application to diagnosis of infections such as acute post-operative osteomyelitis due to its lack of infection specificity [Becker 1998]. Availability of infection specific MRI contrast agents would enable identification of the region of biofilm infection in the context of detailed tissue morphological structure.

The white blood cell scan (WBC) is currently the primary imaging method available to clinicians for diagnosing general infections. WBC is often used to enhance or confirm the interpretation of images obtained by other techniques such as echocardiography [Campeau 1998]. Performing a WBC scan is a laborious procedure that requires ex vivo manipulation and nuclear tagging of white blood cells. To obviate the need of ex vivo manipulation blood cells, methods were developed to specifically target granulocytes in vivo using radiolabeled monoclonal Ab [Gratz 2003]. A relatively new method for following white blood cell migration to sites of inflammation is measurement of consumption of glucose by granulocytes and mononuclear cells using 18F-FDG PET [Koort 2004]. Other alternatives to WBC include radiolabeled non-specific antibodies [Wong 1982; Nijhof 1997] a method that relies on vascular exudation associated with sites of inflammation, and radionuclide three-phase whole body bone imaging which measures increased blood flow to sites of inflammation [Yang 2002]. Radiolabeled chemotactic peptides specifically target sites of infection or inflammation [Rao 2000]. Nonspecific extravascularization of streptavidin at sites of inflammation followed by dosing with a biotin conjugated radionuclide was used to image Staphylococcus aureus (Sa) endocarditis in a rat model [Fogarasi 1999]. The WBC method, as well as the alternative methods mentioned above, detect processes associated with inflammation, and do not discriminate between a sterile inflammation and an infection. One method that may be infection specific rather than inflammatory specific is targeting infections with radiolabeled ciprofloxacin, an antimicrobial agent that binds to bacterial DNA gyrase [Yaper 2001].

Nanoscale platforms offer outstanding systems for engineering the presentation of multiple ligands to optimize infection specificity. The increased permeability of blood vessels at sites of inflammation allows nanocolloids with a diameter less than 100 nm to pass through fenestrations in arterioles and capillaries, enter extravascular space and accumulate at sites of inflammation [De Schrijver 1989]. The advantage of nanocolloids for delivery of imaging agents is that blood clearance is relatively rapid, with a consequent large reduction in background, but the residence time is long enough to allow accumulation at sites of inflammation. Particles that are larger than 100 nm, such as liposomes, are so rapidly cleared from the blood pool that they must be pegylated to be useful [Mulder 2004].

There is a need in the field to develop a non-invasive means for targeting, imaging or detecting and classifying biofilm infections.

### SUMMARY OF THE INVENTION

The present invention is based, in part, on the discovery that certain materials, such as nanoparticles, are useful as therapeutic agents against, or for the imaging of, biofilm infections. Accordingly, the present invention provides compositions and methods useful for the targeting and/or imaging of biofilm infections.

In one embodiment of the invention, it provides a method of targeting a biofilm. The method comprises contacting a biofilm with a composition comprising a protein cage or a protein cage aggregate.

In another embodiment of the invention, it provides a method of imaging a cell, tissue, or biofilm. The method comprises contacting a cell, tissue, or biofilm with a medical imaging composition comprising a protein cage or a protein cage aggregate.

### BRIEF DESCRIPTION OF THE DRAWINGS

FIG. 1 is a TEM showing CCMV-antibody conjugate selectively targeting to Staphylococcus aureus (Sa) cell. Targeted CCMV protein cage (left) and non-targeted control (right).

FIG. 2 shows a) the hexamers and pentamers of a CCMV subunit arranged with icosahedral symmetry, b) a cryoimage of a CCMV cage, and c) the location of sulfhydryls of cys residues incorporated into CCMV; and d) carboxyl groups on surface exposed glu residues of CCMV.

FIG. 3 is an antibody titration curve representing the murine immune response to genetically modified CCMV (right) compared to keyhole limpet hemocyanine (left).

FIG. 4 contains (a) a schematic of Gd coupling to CCMV, (b) LC/MS data verifying coupling, (c) MRM images showing time-course penetration of Gd-coupled CCMV into a representative biofilm, and (d) grey scale MRM images of time-course penetration.

FIG. 5 contains (a) a schematic of the indirect antibody conjugation method for specific targeting of CCMV to S. aureus, (b) flow cytometry results confirming specificity of binding, and (c) TEM thin section confirming the integrity of CCMV bound to the S. aureus cell wall.

FIG. 6 is a schematic showing three possible binding configurations for antibody-conjugated CCMV in the cell wall of S. aureus.

FIG. 7 shows (a) the conjugation schemes for antibody-CCMV linking via indirect conjugation, and (b, c, d) direct conjugation in order of increasing complexity, with options for dual-valency constructs enclosed in dashed-line squares. The bottom contains the legend of symbols used.

FIG. 8 shows (a) a schematic showing constructions of CCMV with different densities of multivalent antibody presentation resulting from mixed reassembly as depicted in FIG. 7a, and (b) constructions of CCMV with dual valent presentation. Symbols are defined in FIG. 7.

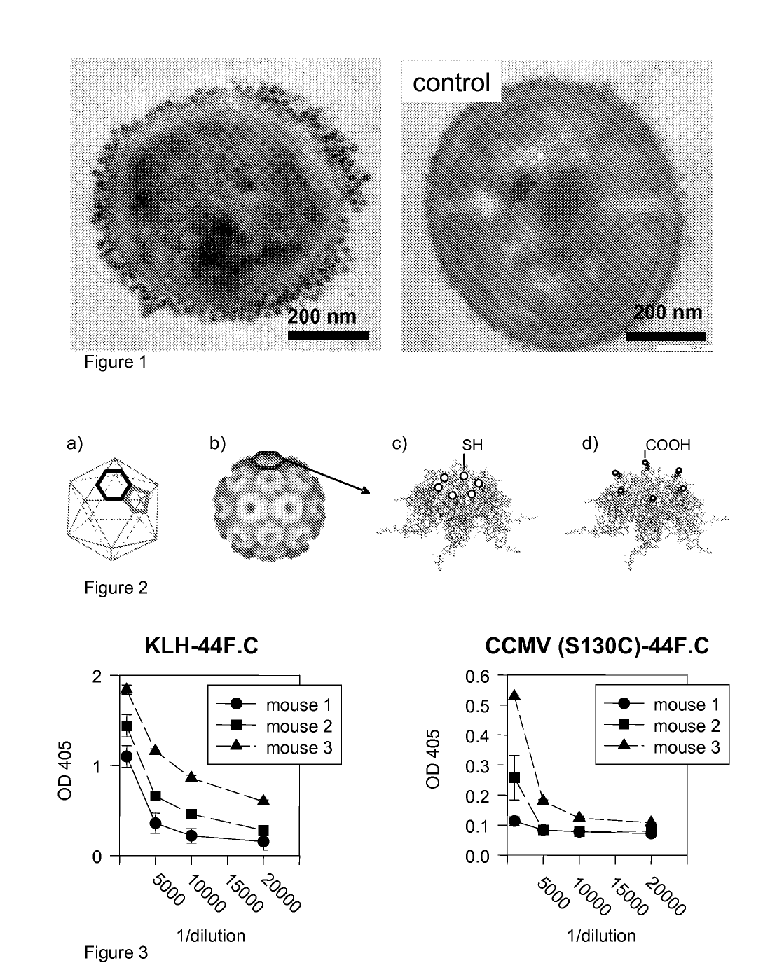

### DETAILED DESCRIPTION OF THE INVENTION

The present invention is based, in part, on the discovery that certain materials, such as nanoparticles, are useful as therapeutic agents against, or for the imaging of, biofilm infections. Accordingly, the present invention provides compositions and methods useful for the targeting and/or imaging of biofilm infections.

According to one aspect of the present invention, it provides a method of targeting a biofilm. The method comprises contacting a biofilm with a composition comprising a protein cage or a protein cage aggregate.

The protein cages or protein cage aggregates can be any cage suitable for targeting a biofilm. In one embodiment, the protein cage comprises a viral, non-viral or bacterial protein. In another embodiment, the protein cage is based on robust platform nanotechnology which uses the self-assembled protein cage architecture derived from viruses and other cage-like architectures for targeted delivery of imaging and antimicrobial agents to biofilm forming bacterial colonies.

The size and monodispersity of many protein cages make them ideal platforms for specific targeting of biofilms with image contrast agents. Advances in nano-engineering have greatly increased our ability to finely tune the presentation of functional groups on selected cages. This expanding capability provides new opportunities to optimize targeting specificity, incorporate antimicrobials into cages and conjugate imaging agents to cages. In one embodiment, the structure and chemistry of protein cages are engineered to construct a family of nanoscale platforms useful for both diagnosing and treating biofilm infections. A distinct advantage of protein cages compared to other nanoparticles is that they provide an array of multifunctional possibilities which can be exploited to optimize the specificity and sensitivity of magnetic resonance imaging (MRI) of loci of biofilm infections.

In another embodiment, the protein cage comprises a virus, e.g., a non-infective plant virus such as cowpea chlorotic mottle virus (CCMV). CCMV is a monodisperse nanoparticle with a diameter of 28 nm. Antibody coupling increases the mean diameter to approximately 52 nm, which falls within the ideal size range to achieve the optimal balance between efficient clearance and vascular exudation. In still another embodiment, CCMV is used for targeted delivery of contrast agents to both more accessible biofilm infections (e.g., vascular graft infections) and those that are more sequestered from the blood pool (osteomyelitis). Research directed at diagnosis and treatment of cancers has paved the way for this approach which essentially falls into the realm of “MR molecular imaging” [Jaffer 2005]. The resolution and sensitivity of MRI images obtained using nanoparticle coupled contrast agents has become sufficient to visualize progenitor stem cell migration using a clinical MRI instrument [Bulte 2001] and smooth muscle monolayers using Pico molar concentrations of nanoparticles [Morawski 2004]. In a two stage reaction, streptavidin-conjugated superparamagnetic nanoparticles were used to target biotinylated monoclonal antibody bound to tumor cells [Artemov 2003]. More recently, Ab-conjugated iron oxide nanoparticles were used to target a receptor present on mammary carcinoma cells lines and visualize the cells using T₂ weighted images [Funovics 2004].

The CCMV protein cage is well suited for specific targeting of a medical imaging contrast agent to sites of biofilm infection as: 1) CCMV has a relatively rigid structure with an interior that is accessible to water; thus contrast agents, such as paramagnetic materials, for example, gadolinium (Gd), obtain a greatly increased relaxivity when incorporated into the cage, e.g., five times that of Gd-coupled dendrimers [Allen 2005]; 2) the known X-ray structure allows CCMV to be used as a scaffold for precise spatial placement of ligands; thus, it can be decorated with multiple conjugates and engineered to optimize binding specificity through multivalent interactions [Speir 1995; Gillitzer 2002]; 3) the modular subunit structure can be exploited to finely tune multivalent ligand presentation; 4) CCMV can be functionalized by genetic manipulation and by a variety of chemical methods [Douglas 2002; Gillitzer 2002]. In this respect it is a highly versatile nanoplatform; 5) the monodispersity should substantially enhance the predictability of hematogenous transport and clearance behavior; and 6) Genetically modified and multiply labeled CCMV preparations maintain their integrity over long periods of storage.

In one embodiment, the protein cage comprises at least one modified subunit. In another embodiment, it comprises at least two modified subunits. In yet another embodiment, the protein cage comprises more than one type of modified subunit, for example, a chemically modified subunit or a genetically modified subunit.

Protein cages are highly symmetrical self-assembled architectures with three distinct interfaces that are amenable for chemical and genetic modification. The outer surface presents an interface on which targeting ligands can be presented. Two complementary approaches have proven successful in the presentation of targeting ligands. The first utilizes the chemical attachment of targeting antibodies or small targeting peptides. This can be achieved through coupling of Ab/peptide through lysine or cysteine residues using bi-functional linkers or through ‘click’ chemistry coupling of attached alkyne and azide moieties. The second approach uses the genetic incorporation of targeting peptides as protein fusions to either the N- (or C-) terminus or inserted into surface exposed loops. In one embodiment, antibodies or antibody fragments are chemically coupled to the protein cage architecture using “click” chemistry reaction.

In another embodiment, the protein cage comprises one or more targeting moieties. In yet another embodiment, the protein cage comprises at least two targeting moieties. Examples of targeting moieties include, but are not limited to, polypeptide targeting moieties or antibody targeting moieties.

Different functional groups on the protein cages can be used for the conjugation and/or loading of various molecules. In one embodiment, the protein cage comprises CCMV, the nanoscale dimensions of which are used for engineering optimal multiple binding functionalities onto a particle that can deliver desired levels, for example, high concentrations, of desired moieties to sites of biofilm infection. In another embodiment, the protein cage comprises CCMV to which antibodies are conjugated and/or a paramagnetic contrast agent is loaded. Such a protein cage can, for example, deliver high concentrations of paramagnetic material to sites of biofilm infection.

In still another embodiment, the protein cage comprises a guest material. Examples of a guest material include, but are not limited to, a therapeutic agent, a medical imaging agent, agents that assist the therapeutic or medical imaging agent and an inorganic material, such as a metal, e.g., a paramagnetic metal. In a further embodiment, the protein cage comprises a linker. Examples of a linker include, but are not limited to, a chelate or a peptide, for example a mineral phase-binding peptide. The mineral phase-binding peptide, can, optionally, further comprise an inorganic material.

The therapeutic agent can be any moiety known to one of skill in the art to be useful for the treatment of a biofilm infection. Examples of therapeutic agents include, but are not limited to, antimicrobial agents, including, reactive oxygen species, photodynamic therapy agents etc. In a further embodiment, the protein cage, protein cage aggregate or the therapeutic agent contained therein can penetrate the targeted biofilm.

The medical imaging agent can be any agent known to one of skill in the art to be useful for imaging a cell, tissue or a biofilm. Examples of medical imaging agent include, but are not limited to, magnetic resonance imaging (MRI) agents, nuclear magnetic resonance imaging (NMR) agents, x-ray agents, optical agents, ultrasound agents and neutron capture therapy agents. The medical imaging agent can be either directly or indirectly coupled to the protein cage. In one embodiment, the medical imaging agent is directly bound to the protein cage through chemical modification of one or more subunits of the protein cage. In another embodiment, the medical imaging agent is indirectly bound to the protein cage through a linker.

The inner surface of the assembled protein cage architecture provides an ideal interface for either covalent or electrostatic attachment of therapeutic and medical imaging agents, which are then encapsulated within the protein cage and therefore sequestered from the exterior environment (e.g. not bioactive against non-targeted cells and tissues). Antimicrobial agents are covalently attached to functional groups uniquely located on the interior surface through cleavable linkers. Antimicrobial agents can be packaged within the cage architecture through interactions (electrostatic, hydrophobic) with the interior surface, using diffusion as a release mechanism. Alternatively, a catalyst capable of producing an antimicrobial agent such as reactive oxygen species (ROS) can be incorporated within the cage either through covalent or electrostatic interactions. Photodynamic therapy agents which are effective for the light driven destruction of microbial biofilms can also be incorporated within the cage.

The Ab-conjugate presentation can be manipulated to optimize the binding specificity of cages to a model biofilm. Affinity of functionalized protein cages for biofilm associated epitopes is enhanced by engineering optimal antigen binding site presentation, while affinity associated with non-specific interactions remains unaffected. As a consequence, it is possible to obtain a high level of specific labeling of biofilms by the cage-bound contrast agent for MR imaging of biofilm infections at a relatively low dosing level. Ab presentation can be manipulated in two ways: by varying the density of presentation of a single Ab, thus altering the possibilities for multivalent binding; and by conjugating two different Ab to the cage, which introduces the possibility of dual valent binding.

The loading of contrast agents can also be manipulated to optimize the sensitivity of cage derived MRI contrast enhancement. Coupling contrast agents to CCMV provides a means to render biofilms highly visible in MR images by enabling delivery of a large amount of contrast agent per binding event. Two approaches can be used to load contrast agent material, e.g., paramagnetic contrast agent material, into the cage: a paramagnetic-chelating agent, for example, a Gd-chelating agent can be covalently bound to the cage; and cages can be mineralized with the contrast agent, e.g., paramagnetic material. In both cases conjugation of the contrast agent and the targeting moiety can be effected through independent functional groups on the cage, thus enabling a substantial amount of contrast agent to be loaded into the cage, while maintaining control over Ab-conjugate presentation.

The invention further provides a protein cage that comprises a reversible switch. The switch can be any switch that controls the structure of the protein cage and operation of the switch changes the physical or chemical nature of the protein cage. In one embodiment, the reversible switch switches the protein cage between a static open state, in which, for example, the protein cage exists in an open or swollen form that allows external material access to its cavity, and a static closed state, in which, for example, the protein cage exists in a closed form that prevents external material from accessing its cavity. In another embodiment, the reversible switch is a pH-dependent switch.

The protein cages of the invention can additionally comprises at least one hydrolase cleavage site. The hydrolase cleavage site can be located in any subunit of the protein cage, or on the interior or the exterior of the protein cage. The hydrolase can be any hydrolase known to one of skill in the art and generally classified as an EC 3 enzyme. In one embodiment, the hydrolase is a protease, e.g., trypsin or cathepsin.

The biofilm infection targeted by the methods of the invention can be any biofilm infection arising from or associated with any now known, or later discovered source. In one embodiment, the biofilm comprises Staphylococcus aureus (Sa) bacteria. Sa is a human pathogen that plays a prominent role in nosocomial infections [USDHHS 1996; Central Public Health Laboratory 2000]. Sa forms biofilms as part of its adaptation to life in the host. It is well adapted to adhere to both tissues and biomaterials coated with blood proteins via a set of adhesins known as microbial surface components recognizing adhesive matrix molecules (MSCRAMM) [Harris 2002; Navarre 1999]. Evidence indicates that Sa expresses biofilm specific genes [Beenken 2003; Beenken 2004; Lim 2004] and exploits cell-to cell communication in biofilm development [Yarwood 2004]. Sa biofilms form readily in vitro and in vivo on biomaterials [Williams 1997; Gracia 1998; Luppens 2002; Kadurugamuwa 2003; Wu 2003]. Compared to other bacterial biofilm-formers, Sa biofilms exhibit resistance to antimicrobial agents that is extraordinary both in terms of the spectrum of antimicrobial agents and the doses that can be tolerated [Olson 2002]. Biomaterial-centered infections are a clear example of biofilm pathogenesis and historically Staphylococcus epidermidis and Sa have been prominent players [Dankert 1986; Gristina 1987]. The prognosis for patients with surgical implants infected with Sa is ominous and removal of the implant is normally recommended [Darouiche 2004]. Sa is known to form biofilms on graft material in vivo [Nigri 2001] and is particularly prominent in infections associated with vascular grafts [Henke 1998; Taylor 2004]. Sa is a primary culprit in prosthetic joint infections, second only to coagulase-negative staphylococcus [Zimmerli 2004]. The projected combined cost for treating infections associated with vascular grafts and prosthetic joint infections in the US is $1.5 billion [Darouiche 2004]. The biofilm model of infection is consistent with symptoms and behavior of chronic infections that involve adhesion and local accumulation of pathogens on tissue, e.g., endocarditis and osteomyelitis [Costerton 1999]. Historically, Sa is the most common pathogen involved in native heart valve infection [Dankert 1986]. Sa is becoming increasingly important in nosocomial endocarditis [Devlin 2004] and endocarditis associated with HIV [Valencia 2004]. Sa is the most common infective agent of hematogenous osteomyelitis [Carek 2001, Dirschl 1994] and vertebral osteomyelitis [Sapico, 1996] and is a frequent player in diabetic foot osteomyelitis [Mader 1996].

In another embodiment, the biofilm arises from a nosocomial or HIV-related infection. In yet another embodiment, the biofilm is associated with an endocarditis-related or osteomyelitis-related infection. In still another embodiment, the biofilm is adhered to tissues or biomaterials, in vitro or in vivo, or the biofilm is adhered to surgical implants or grafted biomaterial. Further, the biofilm can be an antibiotic-resistant biofilm.

According to another aspect of the present invention, it provides a method of imaging a cell, tissue, or biofilm. The method comprises contacting a cell, tissue, or biofilm with a medical imaging composition comprising a protein cage or a protein cage aggregate. In one embodiment, the medical imaging composition penetrates the cell, tissue, or biofilm. In another embodiment, the medical imaging composition further comprises a medical imaging agent such as those described herein. In yet another embodiment, the method further comprising rendering an image of the cell, tissue, or biofilm. The image can be rendered by any imaging technique known to one of skill in the art. Exemplary methods of rendering an image include, but are not limited to, MRI, NMR, x-ray, optical ultrasound and neutron capture therapy.

The present invention also provides protein cages that can mineralize a metal, such as, for example, iron, to form a size-constrained material and/or protein cages that comprise a mineralized inorganic material, e.g., a mineralized metal. In one embodiment, the metal that is mineralized is not iron. Such protein cages may be mineralized under physiological conditions (See Yang, X. et al., Iron oxidation and hydrolysis reactions of a novel ferritin from Listeria innocua. Biochem J. 2000 Aug. 1; 349 Pt 3:783-6; Stefanini, S. et al., Incorporation of iron by the unusual dodecameric ferritin from Listeria innocua. Biochem J. 1999 Feb. 15; 338 (Pt 1):71-75. Erratum in: Biochem J 1999 May 1; 339 (Pt 3):775; Bozzi, M., et al. (1997) A Novel Non-heme Iron-binding Ferritin Related to the DNA-binding Proteins of the Dps Family of Listeria innocua. J. Biol. Chem. 272, 3259-3265) or non-physiological conditions (Allen M. et al., 2002, Protein Cage Constrained Synthesis of Ferrimagnetic Iron Oxide Nanoparticles. Adv. Mater. 14, 1562-1565; Allen, M. et al., (2003). Constrained Synthesis of Cobalt Oxide Nano-Materials In the 12-subunit Protein Cage From Listeria innocua. Inorg. Chem. 42, 6300-6305).

In another embodiment, the present invention provides protein cages formed from Dps proteins that contain a metal mineralized under non-physiological conditions. In one embodiment, the Dps proteins are from L. innocua. Non-physiological conditions include a certain temperature and pH. Exemplary non-physiological conditions include a temperature from about 50° C. to about 85° C. or a pH of about 7.5 to about 9, or a pH of about 6.5. In one embodiment, the temperature may be about 50° C. or greater, about 55° C. or greater, about 60° C. or greater, about 61° C. or greater, about 62° C. or greater, about 63° C. or greater, about 64° C. or greater, about 65° C. or greater, about 70° C. or greater, about 75° C. or greater, about 80° C. or greater, or about 85° C. In another embodiment, the pH may be about 6.5, about 7.5, about 8, about 8.5, or about 9.

### EXAMPLES

### Example 1

### CCMV as an Engineered Multifunctional Nanoparticle

CCMV has become a model system for studies of viral structure and self-assembly [Speir 1995, Zlotnick 2001] (FIG. 2). We have developed means to engineer CCMV both genetically and chemically [Douglas 1998; Gillitzer 2002; Basu 2003; Klem 2003]. We have constructed a library that contains constructs of CCMV that contain single amino acid changes at 32 of its 190 residues, all of which assemble into intact particles. We have developed a variety of protein cages, including CCMV, for targeted drug delivery to tumor cells [Flenniken 2005]. Using site directed mutagenesis cysteines (C) were placed on CCMV in positions that optimize accessibility to functionalization of sulfhydryls via maleimide moieties, while obviating the need for storage in reducing agent to prevent cross-linking of cages via thiol groups. Our ATR-FTIR data show that the biotins present on the surface of CCMV-S-B constructed from S102C are accessible to streptavidin (StAv) and that this conjugation method can be used to link another biomolecule (in this case CCMV-S-B) to CCMV-S-B (data not presented).

As seen in FIG. 2, CCMV is composed of 180 monomer subunits (20 kDa) that self-assemble in vitro into a spherical protein cage 28 nm in diameter. FIG. 2 shows hexamers and pentamers of the subunit that are arranged with icosahedral symmetry (FIG. 2a); a cryo-image of the cage (FIG. 2b); and wireframe renditions of a hexamer [Speir 1995] showing location of sulfhydryls of cys residues incorporated into CCMV-SH (S102C) (FIG. 2c), and carboxyl groups on surface exposed glu residues (FIG. 2d). Positions of residue functional groups are approximately the same for hexamers and pentamers. There are 9 lysines per monomer subunit, at least 3 of which are easily functionalized with NHS esters, and 14 additional carboxyl groups, 3 of which have been functionalized [Gillitzer 2002].

### Example 2

### Immune Response

Based on antibody titer, we showed that the response to CCMV, genetically modified by incorporation of cysteine residues, is slightly more mild than the response to keyhole limpet hemocyanine (KLH), a protein used for vaccine development in humans [Krug 2004] (FIG. 3). These data suggest that the surface of CCMV may not need to be modified to shield it from the host immune response in mammalian systems. The immune response can be further mitigated by surface modification if required [e.g., Raja 2003]. In these experiments the mice exhibited no signs of an extreme immune response such as anaphylactic shock (for which a murine model exists) [Moon 2005]. In contrast, mice injected with M13 bacteriophage exhibited an extreme immune response (two out of three died within 2 weeks).

### Example 3

### MRM of Gd-CCMV Penetration into a Biofilm

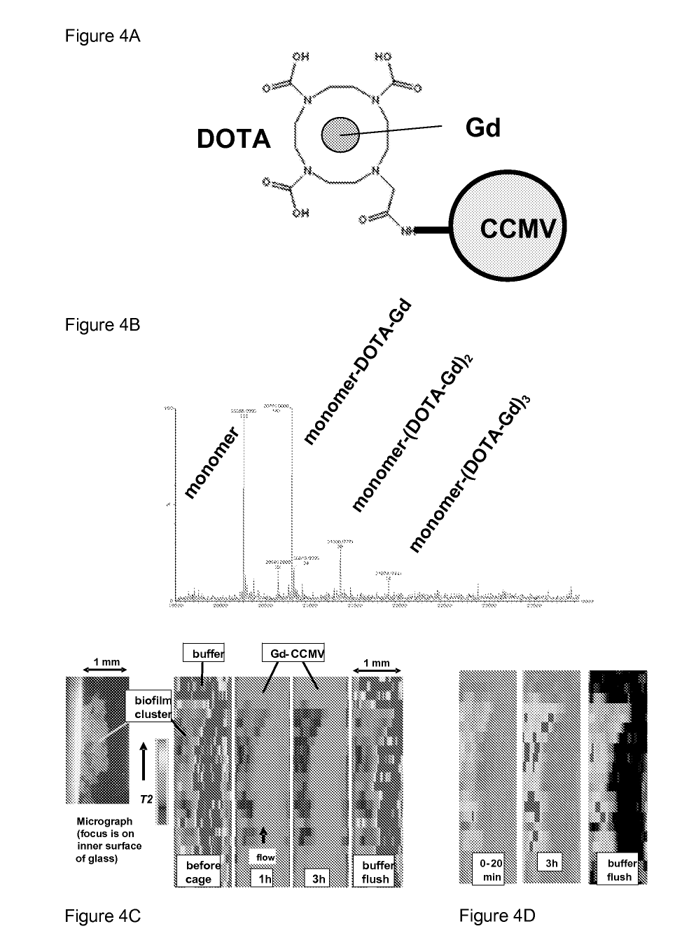

We prepared Gd-DOTA-CCMV in which Gd is coupled via the clinically relevant chelating agent p-NHS-Bn-DOTA (DOTA) (Macrocylics) (FIG. 4a). DOTA was covalently linked to lysine residues (confirmed by LC/MS) (FIG. 4b). MRM was used to image a S. epidermidis biofilm (FIG. 4c).

A schematic of Gd coupling to CCMV via DOTA is shown in FIG. 4a. FIG. 4b shows LC/MS data verifying the coupling. FIG. 4c shows MRM results showing penetration of Gd-DOTA-CCMV into a S. epidermidis biofilm. Biofilm was cultured in TSB in a 1 mm square glass capillary tube and imaged as for a previous study [Seymour 2004]. Biofilm was visible by eye, and the dense nature of the biofilm was evident by transmission microscopy at 100× (left image). MRM images taken before exposure to Gd-DOTA-CCMV and at various times after exposure are on the right, color coded to indicate the T2 values (voxel size 20×156 pm, 300 μm depth). Water has a large T2 value (red) that is diminished by restricted mobility in the biofilm, and then decreased further by the presence of Gd-DOTA-CCMV. Penetration of the cage into more accessible regions of the biofilm occurs within 1 hour (green to blue). Penetration into inner regions of the biofilm is evident at 3 h by a change from blue to purple in certain regions (a 15-20% decrease according to the scale bar). Cage was rinsed from the biofilm much faster than it penetrated, a phenomenon we attribute to convective transport. FIG. 4d shows grey scale images of time points immediately after CCMV injection, the 3 h sample and the sample immediately after the buffer flush. Smaller T₂ values are depicted by brighter pixels. Red areas indicate pixels with brightness exceeding a threshold value (corresponding approximately to a purple hue).

As for other tissue samples, features of the biofilm were distinguishable primarily due to restriction of the water mobility. The biofilm was then exposed to an aqueous solution of Gd-DOTA-CCMV and MRM images acquired periodically. The penetration of Gd-DOTA-CCMV into the biofilm was evident in T₂ maps (FIG. 4c). These data show that Gd-DOTA-CCMV penetrates a relatively dense Staphylococcus biofilm, and that the contrast conferred by Gd-DOTA-CCMV is sufficient for MRI visualization. Gd-DOTA-CCMV was injected into the flow cell at approximately 0.5 ml/min and flow was discontinued after 1 ml. The time course for penetration of the cage into the biofilm is reasonable for transport by diffusion. Approximately 10 min before the last image in the time series in FIG. 4 was acquired, the flow cell was rinsed with 10 ml buffer at a flow rate of 10 ml/min. A likely explanation for the relatively rapid disappearance of the cage from the biofilm is that transport was driven primarily by convection [DeBeer 1994]. A small amount of Gd-DOTA-CCMV was still detected in the biofilm after the buffer flush. These data show that a significant MRI enhancement of the biofilm can be obtained from Gd-CCMV bound to cells via a specific targeting mechanism.

### Example 4

### Specific Targeting of Fluorescein Labeled CCMV to the S. aureus Cell Wall Protein A (SpA)

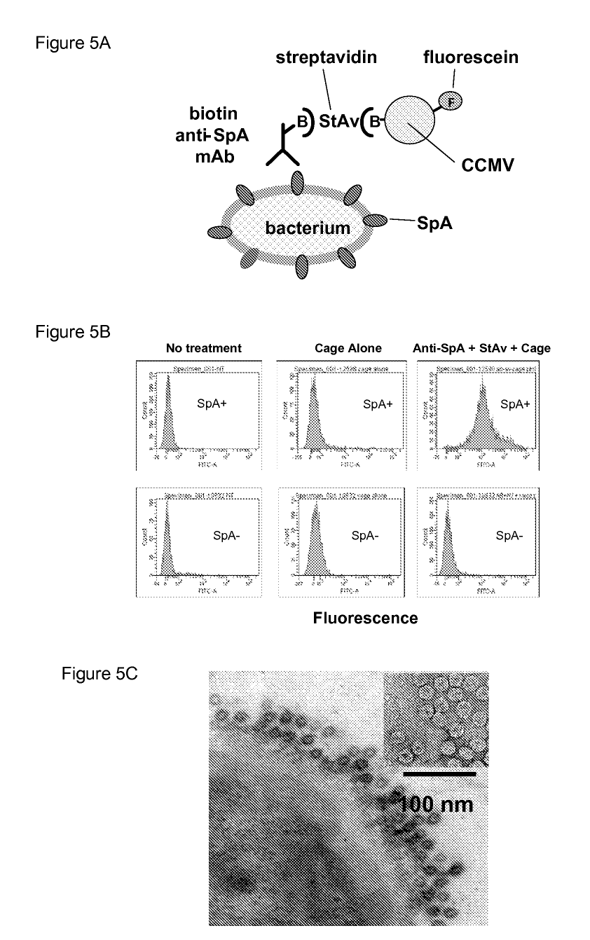

We reproduced previously published flow cytometry results showing that ExtrAvidin-R-Phycoerythrin binding to Cowan I strain via SpA-Ab can serve as a positive control [Wann 1999; Yarwood 2001] and lack of binding to Wood 46 (SpA negative) can serve as a negative control [Wann 1999]. Furthermore, we showed that ATCC strain 29213 is protein A positive when cultured as a colony biofilm. We specifically targeted CCMV to SpA on S. aureus cells using an indirect conjugation method (FIG. 5a), confirmed specific binding using flow cytometry (FIG. 5b), and confirmed integrity of bound CCMV using TEM (FIG. 5c).

FIG. 5 shows a schematic showing progress made in specific targeting of CCMV to S. aureus SpA. FIG. 5a shows an indirect conjugation scheme. FIG. 5b shows flow cytometry results showing that targeting is specifically via SpA; SpA+ is ATCC 12598 (Cowan strain); SpA− is ATCC 10832 (Wood strain). FIG. 5c shows TEM thin section showing intact CCMV bound to the S. aureus cell wall; insert is TEM of CCMV on EM grid (same scale). The cluster formation can be exploited to amplify the enhancement.

### Example 5

### Mixed Reassembly to Regulate the Density of Presentation of a Functional Group

We have shown that reassembly of mixtures of differentially labeled monomer subunits (mixed reassembly) can be used to fabricate CCMV incorporating stoichiometrically controlled densities of two targeting ligands (data not presented).

### Example 6

### Biotinylation of CCMV-SH and Coupling Via Streptavidin

Using attenuated total reflection Fourier transform infrared spectroscopy (ATR-FTIR) data, we have demonstrated coupling of biotinylated CCMV-SH (CCMV-S-B) via streptavidin to surface immobilized CCMV-S-B (data not presented).

### Example 7

### Stabilization of CCMV Via Tyrosine Cross-Linking

We have recently modified a published protocol [Fancy 1999] for cross-linking using tyrosine. SDS-PAGE indicated all monomers in a population of CCMV were crosslinked using this method (data not presented).

### Example 8

### Research Design and Methods

The relationship between bulk concentration of Ab-CCMV (antibacterial antibody conjugated CCMV) and level of binding to biofilms and immobilized ECM (host extracellular matrix proteins) will be obtained. To obtain the biofilm “binding curves” we will exploit the relatively recent development of fast-throughput biofilm reactors that allow the influence of a set of variables to be performed on many “replicate” biofilms grown under identical conditions. Replicates for each condition will allow statistical significance to be evaluated. Ab presentation will be manipulated in two ways:

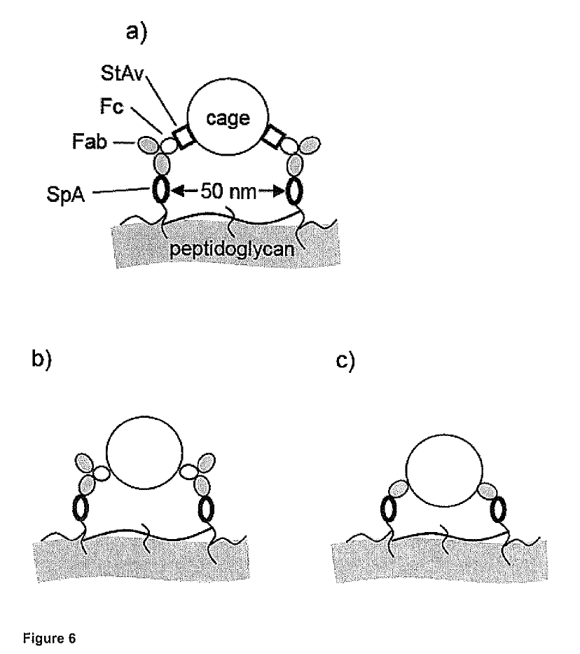

Manipulation 1: CCMV will be conjugated to an anti-protein A (SpA) antibody (SpA-Ab). SpA is the most well characterized Sa surface protein [Harris 2002; Navarre 1999]. The density of presentation of SpA-Ab on CCMV will be varied. The optimal density of multivalent presentation is uncertain, but, without being bound to any theories, a guess is that the average spacing should approximately correspond to the minimum spacing between SpA molecules on the cell surface. In this case maximum multivalent binding will be achieved without interference with binding due to steric hindrance effects. FIG. 6 shows one possible binding configuration for each of three possible Ab-CCMV conjugates. Although the average distance between SpA on the cell surface of wild type Sa is about 500 nm, approximately 20% of SpA are within 50 nm (or less) of each other, a distance within the range of Ab-CCMV conjugates [Harris 2002]. This spatial distribution is undoubtedly in dynamic flux [Navarre 1999].

Manipulation 2: SpA-Ab and an antibody against Sa whole cells (an unspecified component of the cell wall that is not Protein A) (Pg-Ab) will be conjugated to the same cage. This will enable binding of Ab-CCMV to Sa to be mediated both by SpA and epitopes on the adjacent peptidoglycan.

Nanostructures such as CCMV are able to deliver a relatively large amount of paramagnetic material per binding event (e.g., about two orders of magnitude more than a typical Ab). A commercially available nanosized targeted contrast agent will be used as a benchmark to assess the performance of CCMV in this respect.

FIG. 6 shows schematic representations drawn approximately to scale showing possible binding configurations for Ab conjugated CCMV (Ab-CCMV) onto the cell wall of Sa. StAv, streptavidin; SpA, protein A; Fc and Fab segments of the Ab are indicated; FIG. 6a shows indirect conjugation via StAv; FIG. 6b shows direct Ab conjugation (whole Ab); and FIG. 6c shows direct Ab conjugation (Fab′ or Fab).

### Example 9

### Research Methods

### Protein Cages

CCMV-SH (S102C) (see FIG. 2c), wild type (CCMV) and subE will be used for experiments. SubE is CCMV genetic construct in which positively charged N-terminal residues were replaced with negatively charged residues. The negatively charged residues serve to template biomineralization of superparamagnetic iron oxide in the cage interior [Douglas 2002]. Production of assembled CCMV in cowpea plants (CCMV-SH) and constructs such as subE in a yeast host (Pichia pastoris) is routine in the Douglas/Young laboratory [Douglas 1998; Gillitzer 2002; Douglas 2002; Basu 2003; Klem 2003,]. Preparations produced in yeast are devoid of nucleic acids. Both processes can be easily scaled-up for commercial production.

### Bacterial Strains and Culture Conditions

ATCC Se strains 29213, 12598 and 10832 and Pseudomonas aeruginosa PA01 (CBE collection) will be used for experiments. ATCC 29213 is SpA (protein A) positive [Bernardo 2002] and a biofilm former [Harrison 2004]. Cowan I (ATCC 12598) and Wood 46 (ATCC 10832) strains will serve as positive and negative controls for SpA expression, respectively [Wann 1999]. P. aeruginosa PA01 will be used as a negative control in some experiments. Bacteria will be cultured in TSB (tryptic soy broth) at 37° C. [Wann 1999; Schwab 1999; Bollinger 2001; Beenken 2003; Yarwood 2004]. We anticipate that biofilms will reach steady state (growth plus attachment equal to detachment) in approximately 24 h [Lin 2002; Yarwood 2004].

### Antibodies

Both the anti-protein A antibody (SpA-Ab) and the anti-Sa (peptidoglycan) antibody (Pg-Ab) are available commercially in both non-biotinylated and biotinylated form (Sigma-Aldrich products P2921 and B3150, and U.S. biological products S7965-29A and S7965-31). SpA-Ab was developed against the Cowan I strain. Biotinylated and non-biotinylated versions of each these monoclonal Ab are from the same clone. SpA-Ab was used to follow expression of SpA in planktonic cells [Wann 1999; Yarwood 2001] and to identify Sa biofilms on biomedical materials [Belton 2001] and in the lung [Mongodin 2000]. Pg-Ab recognizes SpA negative Sa indicating that its antigen is distinct from SpA. Although a number of putative biofilm specific Ab were identified [Selan 2002; Theilacker 2003; Vancraeynest 2004], none of these are available commercially.

### Ab Conjugation Methods

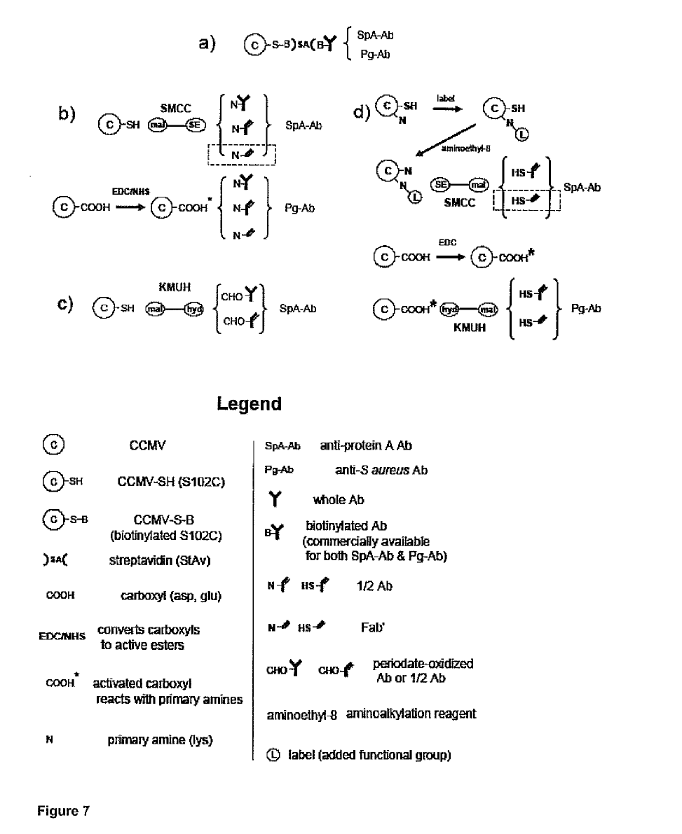

An overview of strategies for conjugation of the two monoclonal anti Sa antibodies (SpA-Ab and Pg-Ab) to CCMV is presented in FIG. 7. The simplest method is indirect conjugation mediated by StAv (FIG. 7a). In order to avoid solution cross-linking, attaching CCMV to Sa by this scheme involves sequentially administering biotinylated-Ab then StAv and finally CCMV-S-B. For clinical applications direct conjugation of Ab to CCMV is desirable since it would entail administration of only a single agent, and, in addition, StAv accumulates non-specifically in areas of inflammation [Fogarasi 1999]. Methods for direct Ab conjugation are presented in order of anticipated level of complexity. We will find one successful direct conjugation method for each Ab (SpA-Ab and Pg-Ab). In all cases SpA-Ab will be attached to CCMV-SH via the free sulflhydryls and Pg-Ab will be attached to CCMV via carboxyl groups (FIG. 2d). All Ab conjugation schemes leave amines free for attachment of the Gd chelating agent or fluorescent label.

Construction and Purification of CCMV with Multivalent and Dual Valent Antigen Binding Site Presentation

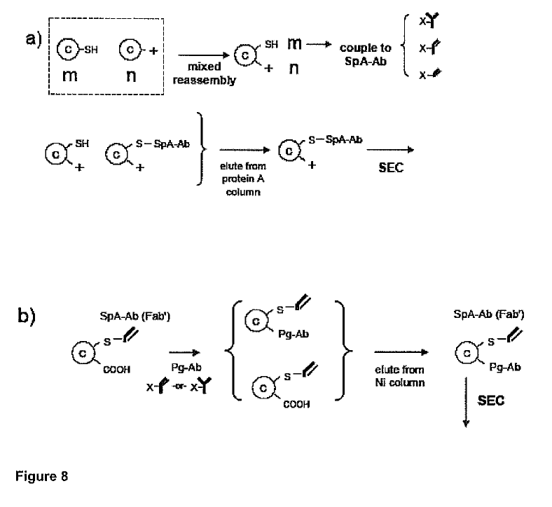

Strategies for constructing and purifying CCMV presenting various densities of SpA-Ab (multivalent presentation) or presenting both SpA-Ab and Pg-Ab (dual valent presentation) are presented in FIG. 8. It is anticipated that a direct conjugation method (FIG. 7 b-d) that preserves the ABS will be found for both Ab.

### Example 10

### Characterization of Biotinylated SpA-Ab (SpA-Ab-B) StAv Mediated Binding of Fluorescently Labeled CCMV-S-B to Sa Planktonic Cells Using Flow Cytometry

The mean fluorescence per cell originating from SpA conferred Ab-CCMV binding to cells using the indirect (StAv mediated) Ab conjugation method (FIG. 7a) will be measured.

Fluorescence per cell obtained with fluorescein labeled CCMV-S-B and fluorescein labeled StAv (StAv-F) will be compared. This will confirm that the simplest binding scheme confers SpA-specific CCMV binding to the test organism (ATCC 29213) in suspension providing a starting point for more complex biofilm studies. In addition, the relative cell-associated fluorescence achieved by fluorescently labeled CCMV-S-B and StAv-F binding to cells will yield an idea of the amplification of MRI signal that can be conferred by the cage

FIG. 7 shows conjugation schemes for linking CCMV to Ab. FIG. 7a shows indirect (StAv mediated) conjugation between biotinylated CCMV-SH (CCMV-S-B) and biotinylated SpA-Ab or Pg-Ab. FIGS. 7b, c, d show alternative methods for direct conjugation in anticipated order of increasing complexity; FIG. 7b shows broken squares indicate desirable direct conjugation options for constructing and purifying CCMV presenting dual valency. SMCC (Pierce product 22322) and sulfo-SMCC (Pierce product 22360) (water soluble) contain maleimide (mal) and succinimidyl ester (SE) groups that link free sulfhydryls with primary amines; EDC and NHS react with carboxyl groups to create an active ester intermediate that reacts with primary amines; if reaction with lysines of Ab does not interfere with the antigen binding site (ABS) then this is probably the simplest direct conjugation scheme for both SpA-Ab (to CCMV-SH) and Pg-Ab (to CCMV carboxyl groups); CCMV carboxyl groups were previously activated with EDC/NHS and reacted with primary amines of functional groups with no cross-linking between CCMV particles [Gillitzer 2002]. Surface exposed carboxyl (Glu) are available on CCMV (FIG. 2d).

FIG. 7c shows a scheme that is an option for SpA-Ab if it is glycosylated (tested using, e.g., Pierce product 23260); periodate-oxidized Ab containing aldehyde groups will react with the hydrazide of KMUH (Pierce product 22111). FIG. 7d shows that Ab can be reduced (and/or reduced and pepsin digested) to produce half Ab (½ Ab) or Fab′, respectively, with exposed sulfhydryls; (the Fab′ will be useful for purification of CCMV presenting dual valency (FIG. 8b)). For conjugation to SpA-Ab, CCMV-SH will be pre-labeled with an amine reactive group (either a fluorescent label or the Gd-chelating agent), then reacted with aminoethyl-8 (N-(iodoethyl) trifluoroacetamide) (Pierce product 23010) that converts free sulfhydryls to amines. EDC activates carboxyl groups to create an active ester intermediate that will react with the hydrazide group of KMUH to form an imide bond; the maleimide of KMUH will react with free sulfhydryls.

FIG. 8 shows construction of CCMV with different densities of multivalent presentation (FIG. 8a) and with dual valent presentation (FIG. 8b). Symbols are essentially the same as in FIG. 7. (+) refers to “wild type” CCMV. Mixed reassembly (FIG. 8a) will be used to construct CCMV having different densities of exposed free sulfhydryls where the mean density is controlled by the input ratio (m:n) of CCMV-SH (S102C) and CCMV(+) monomer subunits. For testing the influence of density of multivalent presentation on CCMV binding to Sa any successful direct conjugation method (FIG. 7b-d) can be used. Purification of excess CCMV from Ab-CCMV will be done using a Protein A affinity column (Pierce product 20356); Size exclusion chromatography (SEC) will be used to remove excess Ab from the purified preparation if necessary.

FIG. 8b shows CCMV with dual valent presentation will be prepared by using CCMV multivalent SpA-Fab′ preparations as starting material; then Pg-Ab (either ½ Ab or whole Ab) will be conjugated to the carboxyl groups using a successful method presented in FIG. 7 b-d. A nickel-chelate affinity column (Pierce product 44920) will be used to purify excess SpA-Ab-CCMV (produced by steps outlined above) from CCMV conjugated to both SpA-Ab and Pg-Ab. CCMV particles remain assembled under elution conditions for the protein A affinity column (0.15 M NaCl, pH 2.8) and nickel-chelate affinity column (0.1 M sodium acetate, pH 5.0) (verified by DLS and TEM) and we previously used a similar method to confer asymmetry on intact CCMV-SH particles (A163C) [Klem 2003]. If the elution conditions for the protein A affinity column disrupt the SpA-Ab ABS the ImmunoPure Gentle Ag/Ab Elution Buffer (Pierce product #21013) will serve as an alternative.

Fluorescent labels and the Gd chelating agent will both be attached via CCMV lysines. Finally, this set of measurements will provide a quantitative comparison of binding between Sa cells and CCMV that can be used as a standard to determine the success of direct conjugation methods. The stoichiometric labeling ratio will be quantified for each CCMV preparation so that this standard of comparison will be evaluated in terms of relative density of Ab-CCMV bound per cell produced by different conjugation techniques.

The positive control will be binding of ExtrAvidin-R-Phycoerythrin to Cowan I strain via SpA-Ab [Warm 1999; Yarwood 2001]. Wood 46 (SpA negative) will be the negative control [Wann 1999]. Level of non-specific binding will be assessed using non-biotinylated CCMV, and by comparing binding with and without pre-exposure of cells to human IgG [Wenn 1999; Yarwood 2001]. Cytograms will be acquired on a BD FACSAria Cytometer with BD FACSAria analysis software (BD Biosciences) to obtain mean fluorescence per cell [Wann 1999]. Fluorescence per cell conferred by binding of fluorescein labeled CCMV-S-B and fluorescein labeled StAv (StAv-F) from a commercial source (e.g., streptavidin, fluorescein conjugate Molecular Probes, S869) will be compared. Labeling ratio of the StAv-F will be obtained spectroscopically. CCMV-S-B will be labeled using published methods [Gillitzer 2002]. Side scattering will be used to quantify cells. Cell DNA will be labeled with a cell permeant fluorescent nuclear stain (SYTO 62, Molecular Probes, S11344, 637/660) [Yarwood 2004; Strathmann 2004]. This will allow definitive identification of scattering events as cells using the multi-channel capability of the flow cytometer. Excitation lasers and emission filters are available on the instrument for each fluor-label (fluorescein (488/515-545), Alexa Fluor 488, R-phycoerythrin (488/564-606) and SYTO 62 (633/650-670)).

### Example 11

### Optimize Binding of Gd Chelating Agent to CCMV-S-B

Gd will be coupled to CCMV-S-B and loading will be optimized. This has been mostly completed using wild type CCMV. We prepared Gd-DOTA-CCMV in which Gd was coupled via the clinically relevant chelating agent p-NHS-Bn-DOTA (DOTA). We will use this same method to couple Gd to CCMV-S-B. Approximately 520 lysines can be labeled using the NHS ester ([Gillitzer 2002]). We will increase the Gd loading further by linking Gd-DOTA to a lysine decamer and then coupling the peptide to CCMV via the N-terminus using the EDC/NHS reaction [Hermanson 1996; Gillitzer 2002]. This will increase the contrast enhancement by 5 to 10 times depending on the rigidity of the CCMV coupled peptide. There is precedent for this chemical modification [Uzgiris 2004].

### Example 12

### Obtain Kinetics of SpA-Ab Conferred Binding of CCMV-S-B to Se Biofilms Using Confocal Scanning Laser Microscopy (CSLM)

Kinetics of interaction of SpA-Ab conjugated CCMV with Sa biofilms will be measured. Indirect conjugation (StAv mediated) will be used to link SpA-Ab-B to CCMV-S-B. Characterization of the kinetics using CSLM sets the stage for binding experiments using CCMV directly conjugated to Ab.

CCMV-S-B will be labeled via the free lysines with Alexa Fluor 488 (quantum yield pH independent from pH 4 to 10). Sa biofilms will be cultured following previous protocols [Pitts 2003; Beenken 2003] in 96 microwell plates with coverglass bottoms (#1.5 German) designed for epi-fluorescence microscopy (Fisher Scientific, 12-566-36). Wells will be inoculated using 200 μl of an overnight batch culture, incubated in the wells for 1 h at 37° C. [Beenken 2003]. Glass wells will be pre-coated with fibronectin to enhance initial adhesion [Pratten 2001]. Plates will be covered and incubated with shaking at 37° C. Every 8-10 h spent medium will be pipetted from wells and replaced with fresh TSB. At 24 h planktonic suspensions and nutrient solutions will be aspirated and wells will be rinsed with buffer and subsequently the blahs will be exposed to buffer containing StAv. A thorough study of the kinetics of penetration and binding of StAv will not be done at this time, but the exposure to StAv will be for a substantial range of time periods (30 min to 4 h). After exposure to StAv the well will be rinsed and the biofilm exposed to SpA-Ab-B (biotinylated SpA-Ab). At various time points the Ab-CCMV solution will be replaced with phosphate buffer (40 mM, pH 7.0) and fixed with 4% paraformaldehyde (30 min) [Zhu 2001]. Cell DNA will be stained and visualized with SYTO 62 (Molecular Probes, S11344, 637/660) [Yarwood 2004; Strathmann 2004].

For 96 wells we will obtain binding time courses having 8 time points in triplicate for biofilms exposed to StAv for 4 different time periods. Extent of fluorescently labeled SpA-Ab-CCMV binding to biofilms will be tracked following a previous method that was used to follow Ab penetration into biofilms [Zhu 2001]. CSLM (Leica TCS-NT or Leica TCS-SP2 AOBS) will be used to obtain optical sections through the entire biofilm in each well at a resolution of 1 pm for three areas in each well using a 63× objective [Redd 2003; Yarwood 2004] (field of view approximately 200×200 μm2). (Plates can be inverted without disturbing the structure of fixed hydrated biofilms). As with flow cytometry, excitation lasers and emission filters for Alexa Fluor 488 and SYTO 62 are available on CSLM instruments. The time for one z-series is approximately 45 s. The estimated time for acquiring data from one well is approximately 10 min so that one set of the triplicate results can be obtained in a reasonable time (approximately 5 h). Since the biofilms will be fixed, each set of the triplicate results can be acquired at a separate session with the plate being kept hydrated at 4° C. between measurements. Data processing will be similar to [Lin 2004] in which the penetration and binding of a fluorescent photoactive oxidant (Merocyanine 540) into Sa biofilms was tracked. For each x-y section of the z-series, fluorescence from bound SpA-Ab-CCMV will be normalized to fluorescence from total cells (nuclear stain). Images will be processed for visualization using Imaris software (Bitplane). Quantitative analysis will be done using MetaMorph software (Universal Imaging Corp.).

### Example 13

### Compare MRI Contrast of Biofilms Obtained with Gd-CCMV-S-B and a Commercially Available Targeted Contrast Agent Using SpA-Ab-B/StAv Mediated Cell Binding

We will assess the performance of CCMV as a delivery vehicle for MRI contrast agent to biofilms. Coupling via DOTA will be used for Gd loading onto CCMV-S-B (Example 11). As in Example 12, indirect conjugation (StAv mediated) will be used to link SpA-Ab-B to Gd-CCMV-S-B.

Biofilms will be cultured in a flow cell compatible with MRI measurements [Seymour 2004] (FIG. 4c). A custom made accessory allows the flow cell (1 mm square glass capillaries) to be placed in the magnet with attached tubing so that biofilms can be exposed to flowing medium in situ. A T-valve regulates flow from either of two sources enabling acquisition of MR images of biofilms before and during exposure to Gd-CCMV-S-B. Biofilms will be cultured in flow cells for 24 h before insertion in the magnet. During this period conventional microscopy will used to track biofilm development. Biofilms will be exposed sequentially to SpA-Ab-B and StAv. Results of Example 12 will be used as a guide to determine times of exposure to SpA-Ab-B and StAv. The flow cell will then be inserted into the magnet. The biotinylated MRI contrast agent will be introduced and its penetration and binding to the biofilm followed. A T₂ image of a biofilm slice (0.3×2.5×20 mm³) can be obtained in 5 min. [Seymour 2004]. This methodology will be used to compare MRI contrast of biofilms obtained with Gd-CCMV (non-biotinylated control), Gd-CCMV-S-B and commercially available 50 nm diameter superparamagnetic biotinylated nanoparticles designed to allow targeted StAv mediated delivery of a T₂ MRI contrast agents to cells (mMACS™ Streptavidin Kit, Invitrogen) [Artemov 2003]. Similar experiments have been performed using Gd coupled to wild type CCMV.

### Example 14

### Conjugate SpA-Ab to CCMV-S Directly and Characterize Binding to Sa Planktonic Cells

We will produce an effective method for direct conjugation of SpA-Ab to CCMV. Direct conjugation of Ab to CCMV will obviate the need for a sequence of injections. In addition to clinical advantages, this approach reduces the complexity of interpretation of results obtained using the in vitro systems.

The simplest direct conjugation will be tried first (FIG. 7b). SpA-Ab (not biotinylated) will be activated by attaching a maleimide group to lysines using SMCC or sulfo-SMCC (conjugation to amines via NHS ester).

Amount of Ab covalently bound to CCMV will be assessed by SDS-polyacrylamide gel electrophoresis (SDS-PAGE) (4-15% Tris-HCI precast gel, Bio-Rad Laboratories) and size exclusion chromatography (SEC) (Bio-Silect 125-5 column, Bio-Rad Laboratories) under conditions in which CCMV dissociates into monomer subunits (100 mM TRIS, pH 7.6). Flow cytometry as outlined in Example 10 will be used to see if the ABS (antigen binding site) is active.

If this relatively simple method does not yield satisfactory results, reactions outlined in FIGS. 7c and d will serve as alternatives, with the reaction outlined in FIG. 7c being the first choice. The presence of carbohydrate on SpA-Ab will be tested before attempting this reaction scheme (Glycoprotein Carbohydrate Estimation Kit, Pierce-see Products folder). SpA-Ab will then be activated with periodate and conjugated using KMUH [Hermanson 1996]. To follow the scheme outlined in FIG. 7d the amines of CCMV-SH will be pre-labeled with either a fluorescent tag or the Gd chelating agent. The free sulfhydryls will then be converted to amines using aminoethylation [Hermanson 1996]. The standard protocol is designed to convert all sulfhydryls (including those involved in disulfide linkages) to amines. Since the sulfhydryls are free and exposed in CCMV-SH there will be no need to denature the proteins with guanidine hydrochloride, and the conversion may proceed at pH 7.2. Otherwise, CCMV-SH will be cross-linked via the tyrosine residues before aminoethylation. Loss of sulfhydryls will be tested using Ellman's reagent (Pierce kit 22582). For all conjugation schemes excess cage will be removed from the reaction mixture by protein A affinity chromatography (FIG. 8a). CCMV remains intact under the recommended elution conditions. If necessary, excess SpA-Ab will be removed by SEC.

### Example 15

### Construct and Characterize CCMV-S with Different Densities of Multivalent Presentation

Ab-CCMV conjugates will be constructed. Mixed reassembly will be used to fabricate CCMV-SH populations presenting a range of desired densities of functional groups (FIG. 8a). CCMV-SH and CCMV (wild type) will be dissembled, mixed in known ratios and reassembled. For disassembly CCMV (wild type or -SH) will be dialyzed into TRIS (pH 7.6) and incubated with CaCl₂ (300 mM), 1 mM TCEP (reducing agent) and RNase A (5μl) for 5 min to remove the ss-RNA. Nucleic acid will be removed by centrifugation and the monomer subunits in the supernatant will be isolated using SEC (Superose-6 gel filtration column equilibrated with 100 mM Tris, pH 7.2, 1 mM TCEP). To induce reassembly, monomer subunits will be incubated in: a) 50 mM sodium citrate (pH 5.25), 1 M NaCl, 1 mM TCEP (2-3 h) and then b) 50 mM sodium citrate (pH 5.25), 50 mM NaCl. Ellman's reagent will be used to measure free sulfhydryls content of mixed reassembly products (Ellman's Reagent, P 22582). SpA-Ab will be conjugated to the mixed reassembly products using a successful direct conjugation method (FIGS. 7b-d). Excess cage will be removed from the reaction mixture by protein A affinity chromatography as in Example 14 and excess SpA-Ab will be removed by SEC if necessary. Amount of Ab covalently bound to CCMV will be determined as in Example 14 for each multivalent preparation. Flow cytometry will be used to assess SpA-Ab mediated CCMV binding to Sa cells using methods outlined in Example 10.

### Example 16

### Characterize Biotinylated Pg-Ab (Pg-Ab-B) StAv Mediated Binding of Fluorescently Labeled CCMV-S-B to Sa Planktonic Cells Using Flow Cytometry

Indirect conjugation (FIG. 7a) will be used to link Pg-Ab-B and CCMV-S-B. Analogous to Example 10, this will confirm that the simplest binding scheme confers Pg-Ab mediated CCMV binding to planktonic ATCC 29213, providing a starting point for assessment of the influence of dual valency on Ab-CCMV binding to biofilms. Methods are as described in Example 10. According to the supplier, the Pg-Ab binds to Se cells that are SpA negative and is species specific. Thus the positive control will be the SpA negative Wood 46 strain and the negative control with be P. aeruginosa PA01.

### Example 17

### Obtain Relationship Between Bulk Concentration and Binding to Biofilms for SpA-Ab-CCMV Possessing Various Densities of SpA-Ab

We will show that optimal multivalent presentation of Ab on CCMV will significantly enhance the binding of CCMV to Sa biofilms, especially at dilute bulk concentrations. It is anticipated that both the kinetics of binding and the saturation (equilibrium) level of binding will be enhanced. The penetration may be inhibited somewhat initially by more tenacious and rapid binding of SpA-Ab-CCMV to Sa cells in the biofilm until saturation of SpA binding sites occurs. Although each multivalent preparation will be composed of a distribution of SpA-Ab-CCMV the mean density will be known and the distribution should be a Poisson distribution.

Kinetics will be characterized (as in Example 12) before acquiring binding data. For characterization of the kinetics we will use the highest practical concentration of CCMV for which no aggregation is observed (approximately 3 mg/mL). The time for 90% saturation for this highest concentration will be used for binding studies. Thus the binding curves will not be equilibrium binding curves for more dilute concentrations. However, the binding curves will still reflect the expected clinical effectiveness of particular multivalent preparation, since in any practical clinical scenario the exposure time is limited.

Fluorescent labeling of Ab-CCMV preparations and biofilm culturing in microwells will be the same as in Example 12. Preparation of biofilms for CSLM will be the same as in Example 12 except that biofilms will not be exposed to StAv. For 96 wells it will be possible to obtain binding time courses having 5 time points in triplicate for 6 different preparations of Ab-CCMV having different densities of Ab (multivalent) presentation. The reasonable time points will be 5, 10, 30, 60 and 120 min.

For binding studies different wells in the 96 microtiter well plate will be used to test the effect of different bulk concentrations of multivalent preparations of SpA-Ab-CCMV on binding to biofilms. Biofilms will be exposed to SpA-Ab-CCMV, prepared for CSLM and viewed using CSLM as for the kinetic study using an exposure time determined in that study. CSLM data will be processed as in Example 12. Thus, the processed data will provide the ratio of CCMV associated fluorescence (Alexa Fluor 488) to cell associated fluorescence (SYTO 62) (SpA-Ab-CCMV binding per cell) for each x-y section of the z series for each biofilm in each well. Using 96 wells the effect of bulk concentration on binding of 6 different multivalent SpA-Ab-CCMV preparations can be tested for 5 different concentrations in triplicate. It is anticipated that a reasonable concentration series will be 3 mg/mL, 1 mg/mL, 0.5 mg/mL, 0.1 mg/mL and 0.05 mg/mL (50 μg/mL) with 6 wells used to obtain the background (no SpA-Ab-CCMV).

The microwell biofilm culture method allows comparison between different multivalent preparations on biofilms grown under identical conditions. To obtain relative Alexa Fluor 488 (SpA-Ab-CCMV) to SYTO 62 (Sa cells) fluorescence values that are meaningful we will: 1) minimize the dependence of fluorescence on solution environment, especially pH. Consequently we will fix the biofilm and rinse with buffer after cage binding and label with a pH insensitive fluor (Alexa Fluor 488); and 2) remain within the linear range of the CSLM instrument by maintaining the PMT gain and laser intensity so that fluorescence is well below saturation.

### Example 18

### Obtain Relationship Between Bulk Concentration and Binding to Immobilized ECM for SpA-Ab-CCMV Possessing Various Densities of SpA-Ab

We will show that non-specific binding to ECM proteins will not be influenced by the density of CCMV Ab presentation. The variable density of surface presenting Ab on multivalent preparations of CCMV precludes using a straightforward ELISA assay to quantify Ab-CCMV binding to immobilized ECM. ATR-FTIR is a surface sensitive spectroscopic technique that is ideal for this study. Using this technique it will be possible to characterize both the immobilized ECM adlayer and the binding of the Ab-CCMV preparations onto this immobilized adlayer. Both kinetics of adsorption and binding curves will be obtained in PBS at pH 7.0 using methods previously developed by us [Suci 1995, 2001b, 2005].

Binding of Ab-CCMV multivalent preparations to ECM proteins fibronectin [Matsuka 2003], fibrinogen [Matsuka 2003] and collagen I [Bowden 2002] will be tested, all of which have been immobilized for various binding assays (protocols are in cited references). By using various surface modification techniques we have characterized protein adsorption onto polymer and amine functionalized surfaces [Suci 1995, Suci 2001c, 2005]. This option will be exploited if it is needed to obtain adequately dense ECM adlayers. Using methods similar to that outlined in Example 12, CSLM microscopy will also be used to characterize binding of fluorescently labeled multivalent Ab-CCMV onto ECM proteins immobilized onto microwells.

### Example 19

### Characterize MRI Contrast of Biofilms Obtained with Multivalent SpA-Ab-CCMV Exhibiting the Best Binding Characteristics

We will assess the improvement in MRI image contrast of biofilms obtained by manipulating CCMV multivalent Ab presentation. Obtaining good biofilm MRI contrast depends on optimizing both sensitivity and specificity. In this case increased specificity will mean that multivalent Ab-CCMV having optimal presentation compete more effectively with the commercial product than Ab-CCMV tested in Example 13. Since the MRI biofilm measurements are relatively time consuming, selected Ab-CCMV exhibiting the best binding characteristics will be chosen from among all the preparations tested thus far.

Gd will be coupled to SpA-Ab-CCMV using the methods presented in Example 11. The methods for MRI measurement to biofilms were presented in Example 13.

### Example 20

### Obtain Relationship Between Bulk Concentration and Binding to Biofilms Cultured in an Annular Reactor for SpA-Ab-CCMV Exhibiting the Best Binding Characteristics

We will assess the influence of biofilm culture conditions on the results obtained thus far. It is well known that biofilms grown under different conditions can exhibit different physiological, biochemical and structural properties. The biofilm annular reactor provides a means to grow biofilms under conditions that contrast sharply with those in the microwells [Goeres 2005]. The annular (CTC) reactor is similar to the rotating disk reactor [Yarwood 2004; Lin 2004] with the advantage that coupons can be inserted or removed during the course of biofilm development. Whereas microwells provide a low shear batch growth environment, the annular reactor creates a high shear, continuous culture environment in which residence time can be controlled independently of shear rate. One disadvantage of this technique is that it is more labor intensive and less amenable to high throughput than the microtiter well method. Therefore, binding of only selected Ab-CCMV to biofilms cultured in annular reactors will be tested. These will be Ab-CCMV preparations that represent the extremes of binding found in the microwell assays thus far. The CTC reactor has 24 coupons enabling a pair of Ab-CCMV preparations to be compared in triplicate following protocols similar to those outlined for Examples 12 and 17. Biofilms will be cultured. Dilution rate will be 0.7 h-1 [Yarwood 2004]. Polycarbonate coupons (1 cm diameter) colonized with Sa biofilm will be removed into 24 well microtiter wells and exposed to Ab-CCMV preparations (as in Examples 12 or 17), and removed into new wells sequentially for rinsing, fixing, and staining with SYTO 62, the nuclear stain, similar to a previous Sa biofilm study [Lin 2004]. Analysis using CSLM will be similar to Examples 12 and 17 except that biofilms will be viewed from the bulk liquid side instead of the base.

### Example 21

### Conjugate Pg-Ab to CCMV-S Directly Via Carboxyl Groups and Characterize Binding to Sa Planktonic Cells

We will develop a method for direct conjugation of Pg-Ab to CCMV that will mediate CCMV binding to Sa cells via an epitope not associated with SpA. Carboxyl groups (FIG. 2d) will be used for direct conjugation of Pg-Ab to CCMV-SH (FIGS. 7b,d). Similar to Example 14 the strategy outlined in FIG. 7b will be tried first. If conjugation to amines of Pg-Ab interferes with the ABS the scheme outlined in FIG. 7d serves as an alternative. As for Example 15, the extent of Ab conjugated onto CCMV will be assessed by SDS-PAGE and SEC. Flow cytometry will be used to see if the ABS of the CCMV conjugated Pg-Ab is active.

### Example 22

### Conjugate SpA-Ab Fab′ to CCMV-S Directly

In order to purify Ab-CCMV possessing dual valency using the strategy outlined in FIG. 8b it is necessary that a form of SpA-Ab be conjugated to CCMV that is lacking the Fc region. Thus, it will be retained on the protein A affinity column but not on the nickel-chelate affinity column. We could use either Fab′ or Fab for this purpose. We will begin by conjugating SpA-Fab′ to CCMV-SH and reserve Fab as an alternative. SpA-Fab′ will be produced by the standard protocol of: 1) digestion of SpA-Ab with immobilized pepsin (Pierce 20343) to obtain F(ab′)₂ [Hermanson 1996] followed by 2) reduction with 2-mercaptoethyliamine to obtain Fab′ [Hermanson 19961. The strategy for conjugation of SpA-Fab′ (or Fab produced by papain digestion) to CCMV will be essentially the same as for the whole SpA-Ab (Example 14).

### Example 23

### Construct and Characterize CCMV Presenting Dual SpA-Ab/P2-Ab Valency

The strategy for producing and purifying CCMV with dual valency is outlined in FIG. 8b. We will characterized Ab-CCMV preparations possessing three different densities of SpA-Ab multivalent presentation (produced in Example 15) will be used as a precursor for the conjugation to Pg-Ab. The three sets of dual valent Ab-CCMV will be fluorescently labeled via the lysines and tested for binding to Sa planktonic cells using flow cytometry as in Example 10.

### Example 24

### Obtain Relationship Between Bulk Concentration and Binding to Biofilms and ECM for CCMV Presenting Dual SpA-Ab/Pg-Ab Valency

Without being bound to any theory, it is possible that, since Pg-Ab will bind cells through antigens that are independent of SpA, Ab-CCMV presenting both Pg-Ab and SpA-Ab will have a greater chance of binding cells through multiple epitopes. This, in turn, will substantially enhance the kinetics and/or saturation binding level of Ab-CCMV binding to Sa biofilms, while non-specific binding to ECM will remain unchanged. The methods we will follow are the same as for Examples 17 (interaction with biofilms) and 18 (interaction with ECM). Binding of the three sets of Ab-CCMV preparations with dual valent presentation prepared in Example 23 will be compared with the corresponding precursor SpA-Ab multivalent preparations (Example 15).

### Example 25

### Characterize MRI Contrast of Biofilms Obtained with Dual and Multivalent SpA-Ab-CCMV Exhibiting the Best Binding Characteristics

This example is similar to Example 19—we will accumulate data indicating how the different Ab-CCMV preparations perform in an in vitro system that is closest to the actual clinically relevant system. As for Example 19, since the MRI biofilm measurements are relatively time consuming, selected Ab-CCMV exhibiting the best binding characteristics will be chosen from among all the preparations tested thus far.

### Example 26

### Compare MRI Contrast Potential of Mineralized subE and a Commercially Available Targeted Contrast Agent

We will assess the performance of CCMV loaded with superparamagnetic iron oxide (SPIO) [Artemov 2003] as an MRI contrast agent. A genetic construct of CCMV (subE) offers the possibility for loading with SPIO. In the case of SPIO loaded subE it would be possible to conjugate biotin or Ab to the cage via lysines. In order to assess the performance of the SPIO mineralized cages, T₁ and T₂ values for bulk preparations of SPIO, a mineralized subE will be compared with bulk preparations of optimally loaded Gd-CCMV obtained in Example 11 and a commercial product (Example 13). SubE will be mineralized by air oxidation of 0.5 mg/mL protein with 25 mM ferrous ammonium sulfate (pH 6.5) [Douglas 2002].

### Example 27

### Alternate Approach for Characterization of the Influence of Multivalent Ab Presentation on Binding of Ab-CCMV to Sa Biofilms

Characterization of the influence of multivalent Ab presentation on binding of Ab-CCMV to Sa biofilms can be achieved even without a suitable direct conjugation by using indirect (StAv mediated) conjugation. we have the tools to proceed with characterization of the influence of multivalent Ab presentation on binding of Ab-CCMV to Sa biofilms (analogous to Examples 16 and 17 above). CCMV-S-B having different densities of biotin will be used as starting material. Both the kinetics and influence of bulk concentration on binding of Ab-CCMV to biofilms will be performed by sequential reaction with SpA-Ab-B, StAv and CCMV-S-B as outlined in Example 12. FIG. 2b shows the approximate dimensions of the configuration for binding to protein A on Sa cells using indirect conjugation. If a direct conjugation method is successful for only SpA-Ab or Pg-Ab, indirect conjugation can be used to determine the influence of dual valent presentation as well.

### REFERENCES CITED

Aaron S D, Ferris W, Remoter K, Vandemheen K, Chan F, Saginur R. (2002) Single and aeruginosa isolates cultured from sputa of adults with cystic fibrosis. J Clin Microbiol. 40:4172-9.
Allen M, Bulte J W, Liepold L O, Basu G, Zywicke H A, Frank J A, Douglas T, Young M. (2005) Paramagnetic viral nanoparticles as potential high-relaxivity magnetic resonance contrast agents. Magn Reson Med. 54(4):807-12.
Amorena B, Gracia E, Monzon M, Leiva J, Oteiza C, Perez M, Alabart J L, Hernandez-Yago J. (1999) Antibiotic susceptibility assay for Staphylococcus aureus in biofilms developed in vitro. J Antimicrob Chemother. 44:43-55.
Anguige K, King J R, Ward J P, Williams P. (2004) Mathematical modeling of therapies targeted at bacterial quorum sensing. Math Biosci. 192:39-83.
Anwar H, Costerton J W. (1990) Enhanced activity of combination of tobramycin and piperacillin for eradication of sessile biofilm cells of Pseudomonas aeruginosa. Antimicrob Agents Chemother. 34:1666-1671.
Anwar H, Biesen T, Dasgupta M, Lam K, Costerton J W. (1989) Interaction of biofilm bacteria with antibiotics in a novel chemostat system. Antimicrob Agents Chemother. 33:1824-1826.
Anwar H, Strap J L, Costerton J W. (1992a) Establishment of aging biofilms: possible mechanism of bacterial resistance to antimicrobial therapy. Antimicrob Agents Chemother. 36:1347-1351.
Anwar H, Strap J L, Costerton J W. (1992b) Kinetic interactions of biofilm cells of Staphylococcus aureus with cephalexin and tobramycin in a chemostat system. Antimicrob Agents Chemother. 36:890-893.
Artemov D, Mon N, Okollie B, Bhujwalla Z M. (2003) MR molecular imaging of the Her-21neu receptor in breast cancer cells using targeted iron oxide nanoparticles. Magn Reson Med. 49:403-8.
Basu G, Allen M, Willits D, Young M, Douglas T. (2003) Metal binding to cowpea chlorotic mottle virus using terbium(III) fluorescence. J Biol Inorg Chem. 8:721-725.
Becker W. (1998) Bone and soft tissue, in THE IMAGING OF INFECTION AND INFLAMMATION; Kuwer Academic Pub, London; Cox P H, Buscombe J R, eds.; pp. 199-217.
Beenken K E, Blevins J S, Smeltzer M S. (2003) Mutation of sarA in Staphylococcus aureus limits biofilm formation. Infect Immun. 71:4206-11.
Beenken K E, Dunman P M, McAleese F, Macapagal D, Murphy E, Projan S J, Blevins J S, Smeltzer M S. (2004) Global gene expression in Staphylococcus aureus biofilms. J Bacteriol. 186:4665-84.
Behr T M, Behe M, Stabin M G, Wehrmann E, Apostolidis C, Molinet R, Strutz F, Fayyazi A, Wieland E, Gratz S, Koch L, Goldenberg D M, Becker W. (1999) High-linear energy transfer. (LET) alpha versus low-LET beta emitters in radioimmunotherapy of solid tumors: therapeutic efficacy and dose-limiting toxicity of 213Bi- versus 90Y-labeled C017-1A Fab′ fragments in a human colonic cancer model. Cancer Res. 59:2635-43.
Belton J M, G-Honduvilla N, Jurado F, Carranza A, Bujan J. (2001) In vitro interaction of bacteria with polypropylene/ePTFE prostheses. Biomaterials. 22:2021-4.
Bernardo K, Pakulat N, Macht M, Krut O, Seifert H, Fleer S, Hunger F, Kronke M. (2002) Identification and discrimination of Staphylococcus aureus strains using matrix-assisted laser desorption/ionization-time of flight mass spectrometry. Proteomics. 2:747-53.
Bollinger N, Hassett D J, Iglewski B H, Costerton J W, McDermott T R. (2001) Gene expression in Pseudomonas aeruginosa: evidence of iron override effects on quorum sensing and biofilm-specific gene regulation. J Bacteriol. 183:1990-6.
Bowden M G, Visai L, Longshaw C M, Holland K T, Speziale P, Hook M. (2002) Is the GehD lipase from Staphylococcus epidermidis a collagen binding adhesin? J Biol Chem. 277:43017-23.
Bulte J W, Douglas T, Witwer B, Zhang S C, Strable E, Lewis B K, Zywicke H, Miller B, van Gelderen P, Moskowitz B M, Duncan D, Frank J A. (2001) Magnetodendrimers allow endosomal magnetic labeling and in vivo tracking of stem cells. Nature Biotech. 19:1141-1147
Campeau R J, Ingram C. (1998) Perivalvular abscess complicating infective endocarditis: complementary role of echocardiography and indium-111-labeled leukocytes. Clin Nucl Med. 23:582-4.
Carek P J, Dickerson L M, Sack J L. (2001) Diagnosis and management of osteomyelitis. Am Fam Physician. 63:2413-20.
Central Public Health Laboratory. (2000) Surviellance of surgical site infections-in English hospitals 1997-1999 UK. (combination antibiotic susceptibilities of planktonic, adherent, and biofilm-grown Pseudomonas)
Costerton J W, Stewart P S, Greenberg E P. (1999) Bacterial biofilms: a common cause of persistent infections. Science. 284:1318-22
Dankert J, Hogt A H, Feijen J. (1986) Biomedical polymers: bacterial adhesion, colonization and infection. Crit Rev Biocompatibility. 2:219-301
Darouiche R O. (2004) Treatment of infections associated with surgical implants. N Engl J Med. 350:1422-9.
Davies D. (2003) Understanding biofilm resistance to antibacterial agents. Nat Rev Drug Discov. 2:114-22.
Davies D G, Parsek M R, Pearson J P, Iglewski B H, Costerton J W, Greenberg E P. (1998) The involvement of cell-to-cell signals in the development of a bacterial biofilm. Science. 280:295-8
Debeer D, Stoodley P, Lewandowski Z. (1994) Liquid flow in heterogeneous biofilms. Biotechnol Bioeng. 44:636-641.
De Kievit T R, Gillis R, Marx S, Brown C, Iglewski B H. (2001) Quorum-sensing genes in Pseudomonas aeruginosa biofilms: their role and expression patterns. Appl Environ Microbiol. 67:1865-73
De Schrijver M. (1989) SCINTIGRAPHY OF INFLAMMATION WITH NANOMETER-SIZED COLLOIDAL TRACERS, Kluwer Academic Pub, London, p. 212.
Devlin R K, Andrews M M, von Reyn C E. (2004) Recent trends in infective endocarditis: influence of case definitions. Curr Opin Cardiol. 19:134-9.
Dinges M M, Orwin P M, Schlievert P M. (2000) Exotoxins of Staphylococcus aureus. Clin Microbiol Rev. 13(1): 16-34.
Dirschl D R. (1994) Acute pyogenic osteomyelitis in children. Orthop Rev. 23:305-12.
Douglas T, Strable E, Willits D, Aitouchen A, Libera M. (2002) Protein Engineering of a Viral Cage for Constrained Nanomaterials Synthesis. Adv Mater Deerfield. 14:415-18
Douglas T, Young M. (1998) Host-guest encapsulation of materials by assembled virus protein cages. Nature. 398: 152-155.
Dubin G. (2002) Extracellular proteases of Staphylococcus spp. J Biol Chem. 383:1075-86.
Fancy D A, Kodadek T. (1999) Chemistry for the analysis of protein-protein interactions: rapid and efficient cross-linking triggered by long wavelength light. Proc Natl Acad Sci U.S.A. 96:6020-4.
FDA. (2005) Introduction to instructions for investigative new drug application
Flenniken M L, Liepold L O, Crowley B E, Willits D A, Young M J, Douglas T. (2005) Selective attachment and release of a chemotherapeutic agent from the interior of a protein cage architecture. Chem Commun (Camb). 2005 Jan. 28; (4):447-9.
Fogarasi M, Pullman J, Winnard P Jr, Hnatowich D J, Rusckowski M. (1999) Pretargeting of bacterial endocarditis in rats with streptavidin and 111In-labeled biotin. J Nucl Med. 40:484-90
Foster J S, Kolenbrander P E. (2004) Development of a multispecies oral bacterial community in a saliva-conditioned flow cell. Appl Environ Microbiol. 70:4340-8.
Fouts T R, Tuskan R G, Chada S, Hone D M, Lewis G K. (1995) Construction and immunogenicity of Salmonella typhimurium vaccine vectors that express HIV-1 gp120. Vaccine. 13:1697-1705.
Funovics M A, Kapeller B, Hoeller C, Su H S, Kunstfeld R, Puig S, Macfelda K. (2004) MR imaging of the her2/neu and 9.2.27 tumor antigens using immunospecific contrast agents. Magn Reson Imaging. 22:843-50.
Gillitzer E, Willits D, Young M, Douglas T. (2002) Chemical modification of a viral cage for multivalent presentation. Chem Commun (Camb.) 21:2390-1.
Goeres D M, Loetterle L R, Hamilton M A, Murga R, Kirby D W, Donlan R M. (2005). Statistical assessment of the ruggedness of a microbiological laboratory method: the CDC biofilm reactor. Microbiology. 151 (Pt 3):757-62.
Gracia E, Lacleriga A, Monzon M, Leiva J, Oteiza C, Amorena B. (1998) Application of a rat osteomyelitis model to compare in vivo and in vitro the antibiotic efficacy against bacteria with high capacity to form biofilms. J Surg Res. 79:146-53.
Gratz S, Schipper M L, Dorner J, Hoffken H, Becker W, Kaiser J W, Behe M, Behr T M. (2003) LeukoScan for imaging infection in different clinical settings: a retrospective evaluation and extended review of the literature. Clin Nucl Med. 28:267-76.
Gristina A G. (1987) Biomaterial-centered infection: microbial adhesion versus tissue integration. Science. 237:1588-95.
Harmsen A G. (2005) Veterinary Molecular Biology, Montana State University, Bozeman Mont. 59717; unpublished results obtained in collaborative research with Mark Young and Trevor Douglas supported by funds from NIH. (NHLBI Programs of Excellence in Nanotechnology)
Harris L G, Foster S J, Richards R G. (2002) An introduction to Staphylococcus aureus, and techniques for identifying and quantifying S. aureus adhesins in relation to adhesion to biomaterials: review. Eur Cell Mater. 4:39-60.
Harrison J J, Ceri H, Stremick C, Turner R J. (2004) Differences in biofilm and planktonic cell mediated reduction of metalloid oxyanions. FEMS Microbiol Lett. 235:357-62.
Henke P K, Bergamini T M, Rose S M, Richardson J D. (1998) Current options in prosthetic vascular graft infection. Am Surg. 64:39-45; discussion 45-6.
Hennequin C, Janoir C, Barc M C, Collignon A, Karjalainen T. (2003) Identification and characterization of a fibronectin-binding protein from Clostridium difficile. Microbiology. 149(Pt 10):2779-87.
Hermanson G T. (1996) BIOCONJUGATE TECHNIQUES, Academic Press, NY. p. 104. (aminoethylation), p. 123. (hydrazide reaction with EDC activated carboxylates), p. 138. (optimal reaction conditions for isothiocyanates), p. 475. (activation of Ab with sodium periodate), p. 463. (production of Fab′), p. 477. (conjugation of CHO-Ab to hydrazide), p. 479. (production of F(abi))
Jaffer F A, Weissleder R. (2005) Molecular imaging in the clinical arena. J Am Med Assoc. 293:855-862.
Jevtic V. (2004) Diagnosis and treatment of diabetic foot infections. Clin Infect Dis. 39:885-910
Kadurugamuwa J L, Sin L V, Yu J, Francis K P, Kimura R, Purchio T, Contag P R. (2003) Rapid direct method for monitoring antibiotics in a mouse model of bacterial biofilm infection. Antimicrob Agents Chemother. 47:3130-7.
Klem M T, Willits D, Young M, Douglas T. (2003) 2-D array formation of genetically engineered viral cages on Au surfaces and imaging by atomic force microscopy. J Am Chem Soc. 125:10806-10807
Koort J K, Makinen T J, Knuuti J, Jalava J, Aro H T. (2004) Comparative 18F-FDG PET of experimental Staphylococcus aureus osteomyelitis and normal bone healing. J Nucl Med. 45:1406-11
Kumon H, Ono N, tide M, Nickel J C. (1995) Combination effect of fosfomycin and ofloxacin against Pseudomonas aeruginosa growing in a biofilm. Antimicrob Agents Chemother. 39: 1038-44.
Lane L C. (1974) The bromoviruses. Adv Virus Res. 19:151-220.
Lequette Y, Greenberg E P. (2005) Timing and localization of rhamnolipid synthesis gene expression in Pseudomonas aeruginosa biofilms. J Bacteriol. 187:37-44
Nigri G R, Tsai S, Kossodo S, Waterman P, Fungaloi P, Hooper D C, Doukas A G, LaMuraglia G M. (2001) Laser-induced shock waves enhance sterilization of infected vascular prosthetic grafts. Lasers Surg Med. 29:448-54.
Lewis K. (2001) Riddle of biofilm resistance. Antimicrob Agents Chemother. 45:999-1007
Lim Y, Jana M, Luang T T, Lee C Y. (2004) Control of glucose- and NaCI-induced biofilm formation by rbf in Staphylococcus aureus. J Bacteriol. 186:722-9.
Lin H Y, Chen C T, Huang C T. (2004) Use of merocyanine 540 for photodynamic inactivation of Staphylococcus aureus planktonic and biofilm cells. Appl Environ Microbiol. 70:6453-8.
Lipsky B A, Berendt A R, Deery H G, Embil J M, Joseph W S, Karchmer A W, LeFrock J L, Lew D P, Mader J T, Norden C, Tan J S. (2004) Diagnosis and treatment of diabetic foot infections. Clin Infect Dis. 39:885-910.
Luppens S B, Reij M W, van der Heijden R W, Rombouts F M, Abee T. (2002) Development of a standard test to assess the resistance of Staphylococcus aureus biofilm cells to disinfectants. Appl Environ Microbiol. 68:4194-200.
Mader J T, Ortiz M, Calhoun J H. (1996) Update on the diagnosis and management of osteomyelitis. Clin Podiatr Med Surg. 13:701-724.
Matsuka Y V, Anderson E T, Milner-Fish T, Ooi P, Baker S. (2003) Staphylococcus aureus fibronectin-binding protein serves as a substrate for coagulation factor XIIIa: evidence for factor XIIIa-catalyzed covalent cross-linking to fibronectin and fibrin. Biochemistry. 42:14643-52.
Mongodin E, Bajolet O, Hinnrasky J, Puchelle E, de Bentzmann S. (2000) Cell wall-associated protein A as a tool for immunolocalization of Staphylococcus aureus in infected human airway epithelium. J Histochem Cytochem. 48:523-34.
Monzon M, Garcia-Alvarez F, Lacleriga A, Amorena B. (2002) Evaluation of four experimental osteomyelitis infection models by using precolonized implants and bacterial suspensions. Acta Orthop Scand. 73:11-9.
Morawski, A M, P M Winter, K C Crowder, S D Caruthers, R W Fuhrhop, M J Scott, J D Robertson, D R Abendschein, G M Lanza, and S A Wickline. (2004) Targeted nanoparticles for quantitative imaging of sparse molecular epitopes with MRI. Magn Reson Med. 51:480-486.
Mulder W J, Strijkers G J, Griffloen A W, van Bloois L, Molema G, Storm G, Koning G A, Nicolay K. (2004) A liposomal system for contrast-enhanced magnetic resonance imaging of molecular targets. Bioconjug Chem. 15:799-806.
Murphy J G, Steckelberg J M. (1995) New developments in infective endocarditis. Curr Opin Cardiol. 10:150-4.
Navarre W W, Schneewind O. (1999) Surface proteins of gram-positive bacteria and mechanisms of their targeting to the cell wall envelope. Microbiol Mol Biol Rev. 63:174-229.
Nigri G R, Tsai S, Kossodo S, Waterman P, Fungaloi P, Hooper D C, Doukas A G, LaMuraglia G M. (2001) Laser-induced shock waves enhance sterilization of infected vascular prosthetic grafts. Lasers Surg Med. 29:448-54.
Nijhof M W, Oyen W J, van Kampen A, Claessens R A, van der Meer J W, Corstens F H. (1997) Evaluation of infections of the locomotor system with indium-111-labeled human IgG scintigraphy. J Nucl Med. 38: 1300-5.
Oldendorf W, Oldendorf W, Jr. (1987) BASICS OF MAGNETIC RESONANCE IMAGING. Martinius Nijhoff Pub, Boston p. 125-6.
Olson M E, Ceri H, Morck D W, Buret A G, Read R R. (2002) Biofilm bacteria: formation and comparative susceptibility to antibiotics. Can J Vet Res. 66:86-92.
Pitts B, Hamilton M A, Zelver N, Stewart P S. (2003) A microtiter-plate screening method for biofilm disinfection and removal. J Microbiol Meth. 54:269-76.
Pratten J, Foster S J, Chan P F, Wilson M, Nair S P. (2001) Staphylococcus aureus accessory regulators: expression within biofilms and effect on adhesion. Microbes Infect. 3:633-7.
Read I, Chatzinikolaou I, Chaiban G, Hanna H, Hachem R, Dvorak T, Cook G, Costerton W. (2003) In vitro and ex vivo activities of minocycline and EDTA against microorganisms embedded in biofilm on catheter surfaces. Antimicrob Agents Chemother. 47:3580-5.
Rao P S, Miele V R, Vassileva-Belnikolovska D, Jungkind D, Thakur M. (2000) A receptor-specific peptide for imaging infection and inflammation. Nucl Med Commun. 21:1063-70.
Raja K S, Wang Q, Gonzalez M J, Manchester M, Johnson J E, Finn M G. (2003) Hybrid virus-polymer materials. 1. Synthesis and properties of PEG-decorated cowpea mosaic virus. Biomacromolecules 4:472-6.
Ruoslahti E. (2005) Cancer Research Center, The Burham Institute, La Jolle Calif. 92037; unpublished results obtained in collaborative research with Mark Young and Trevor Douglas supported by funds from the Office of Naval Research, Center for Bioinspired Nanomaterials Grant
Sakakibara H, Tamura T, Suzuki T, Hisano T, Abe S, Murata K. (2002) Preparation and properties of alginate lyase modified with polyethylene glycol). J Pharm Sci. 91:1191-9.
Sapico F L. (1996) Microbiology. and antimicrobial therapy of spinal infections. Orthop Clin North Am. 27:9-13.
Sauer K, Camper A K, Ehrlich G D, Costerton J W, Davies D G. (2002) Pseudomonas aeruginosa displays multiple phenotypes during development as a biofilm. J Bacteriol. 184:1140-54
Schwab U, Gilligan P, Jaynes J, Henke D. (1999) In vitro activities of designed antimicrobial peptides against multidrug-resistant cystic fibrosis pathogens. Antimicrob Agents Chemother. 43:1435-40.
Scripps. (2005) Website: mmtsb.scripps.edulviperl; X-ray entries>1 cwp Cowpea chlorotic mottle virus>contact tables>edit>search>. (find “K”; only contact is between K131 & Y190)
Selan L, Passariello C, Rizzo L, Varesi P, Speziale F, Renzini G, Thaller M C, Fiorani P, Rossolini G M. (2002) Diagnosis of vascular graft infections with antibodies against staphylococcal slime antigens. Lancet. 359:2166-8.
Seymour J D, Codd S L, Gjersing E L, Stewart P S. (2004) Magnetic resonance microscopy of biofilm structure and impact on transport in a capillary bioreactor. J Magn Reson. 167:322-7.
Spartera C, Morettini G, Baffle G, Di Cesare E, Alagia G, Ventura M. (1997) Diagnostic imaging techniques in vascular graft infection. Eur J Vasc Endovasc Surg. 14(Suppl. A):24-6.
Speir J A, Sanjeev M, Wang G, Baker T S, Johnson J E. (1995) Structures of the native and swollen forms of cowpea chlorotic mottle virus determined by X-ray crystallography and cryo electron microscopy. Structure. 3:63-78.
Stewart P S, Costerton J W. (2001) Antibiotic resistance of bacteria in biofilms. Lancet. 358: 135-8.
Strathmann M, Wingender J. (2004) Use of an axonal dye in combination with confocal laser scanning microscopy to monitor damage to Staphylococcus aureus cells during colonisation of silver-coated vascular grafts. Int J Antimicrob Agents. 24:234-40.
Suci P A, Geesey G G. (1995) Investigation of alginate binding to germanium and polystyrene substrata conditioned with mussel adhesive protein. J Coll Int Sci. 172: 347-357.
Suci P A, Geesey G G, Tyler B J. (2001a) Integration of Raman microscopy, differential interference contrast microscopy, and attenuated total reflection Fourier transform infrared spectroscopy to investigate chlorhexidine spatial and temporal distribution in Candida albicans biofilms. J Microbiol Meth. 46:193-208.
Suci P A, Geesey G G. (2001b) Use of attenuated total internal reflection Fourier transform infrared spectroscopy to investigate interactions between Mytilus edulis foot proteins at a surface. Langmuir. 17:2538-2540.
Suci P A, Geesey G G. (2001c) Comparison of adsorption behavior of two Mytilus edulis foot proteins on three surfaces. Colloids Surf B Biointerfaces. 22: 159-168.
Suci P A, Tyler B J. (2002) Action of chlorhexidine digluconate against yeast and filamentous forms in an early-stage Candida albicans biofilm. Antimicrob Agents Chemother. 46:3522-3531.
Suci P A, Tyler B J. (2003) A method for discrimination of subpopulations of Candida albicans biofilm cells that exhibit relative levels of phenotypic resistance to chlorhexidine. J Microbiol Meth. 53:313-325.
Suci P A, Klem M T, Douglas T, Young M. (2005) Influence of electrostatic interactions on the surface adsorption of a viral protein cage. Langmuir. 21:8686-8693.
Taylor M D, Napolitano L M. (2004) Methicillin-resistant Staphylococcus aureus infections in vascular surgery: increasing prevalence. Surg Infect (Larchmt.) 5:180-7.
Teintze M. (2003) Department of Chemistry and Biochemistry, Montana State University, Bozeman Mont. 59717, mteintze@montana.edu, The original purpose of these experiments was ascertain the potential of CCMV to be used as a carrier in humans to illicit an immune response to HIV antigens for vaccine development. In this respect CCMV performed poorly since it did not illicit a strong response even with added adjuvant. (CFA).
Theilacker C, Coleman F T, Mueschenborn S, Llosa N, Grout M, Pier G B. (2003) Construction and characterization of a Pseudomonas aeruginosa mucoid exopolysaccharide-alginate conjugate vaccine. Infect Immun. 71:3875-84.
Tyson G W, Chapman J, Hugenholtz P, Allen E E, Ram R J, Richardson P M, Solovyev W, Rubin E M, Rokhsar D S, Banfield J F. (2004) Community structure and metabolism through reconstruction of microbial genomes from the environment. Nature. 428:37-43.
USDHHS. (1996) Centers for Disease Control and Prevention. National Nosocomial Infection Surveillance System report: data summary from October 1986-April 1996. Atlanta. (Ga.)
Uzgiris E E, Cline H, Moasser B, Grimmond B, Amaratunga M, Smith J F, Goddard G. (2004) Conformation and structure of polymeric contrast agents for medical imaging. Biomacromolecules. 5:54-61.
Valencia E, Miro J. (2004) Endocarditis in the setting of HIV infection. AIDS Rev. 6:97-106.
Vancraeynest D, Hermans K, Haesebrouck F. (2004) Genotypic and phenotypic screening of high and low virulence Staphylococcus aureus isolates from rabbits for biofilm formation and MSCRAMMs. Vet Microbiol. 103:241-7.
Wann E R, Fehringer A P, Ezepchuk N, Schlievert P M, Bina P, Reiser R F, Hook M M, Leung D Y. (1999) Staphylococcus aureus isolates from patients with Kawasaki disease express high levels of protein A. Infect Immun. 67:4737-43.
Ward K H, Olson M E, Lam K, Costerton J W. (1992) Mechanism of persistent infection associated with peritoneal implants. J Med Microbiol. 36:406-13.
Werner E, Roe F, Bugnicourt A, Franklin M J, Heydorn A, Morin 8, Pitts B, Stewart P S. (2004) Stratified growth in Pseudomonas aeruginosa biofilms. Appl Environ Microbiol. 70:6188-96.
Whiteley M, Bangera M G, Bumgarner R E, Parsek M R, Teitzel G M, Lory S, Greenberg E P. (2001) Gene expression in Pseudomonas aeruginosa biofilms. Nature. 413:860 4
Williams I, Venables W A, Lloyd D, Paul F, Critchley I. (1997) The effects of adherence to silicone surfaces on antibiotic susceptibility in Staphylococcus aureus. Microbiology. 143:2407-13.
Williamson M R, Boyd C M, Shah H R. (1989) Prosthetic vascular graft infections: diagnosis and treatment. Crit Rev Diagn Imaging. 29:181-213.
Wolfbarst A B. (1999) LOOKING WITHIN, University of CA Press, p. 21.
Wong D W, Dhawan V K, Tanaka T, Mishkin F S, Reese I C, Thadepalli H. (1982) Imaging endocarditis with Tc-99m-labeled antibody—an experimental study: concise communication. J Nucl Med. 23:229-34.
Wu J A, Kusuma C, Mond J J, Kokai-Kun J F. (2003) Lysostaphin disrupts Staphylococcus aureus and Staphylococcus epidermidis biofilms on artificial surfaces. Antimicrob Agents Chemother. 47:3407-14.
Wunderbaldinger P, Josephson L, Bremer C, Moore A, Weissleder R. (2002) Detection of lymph node metastases by contrast-enhanced MRI in an experimental model. Magn Res Med. 47:292-297.
Yang D C, Ratani R S, Mittel P K, Chua R S, Pate S M. (2002) Radionuclide three-phase whole-body bone imaging. Clin Nucl Med. 27:419-26
Yapar Z, Kibar M, Yapar A F, Togrul E, Kayaselcuk U, Sarpel Y. (2001) The efficacy of technetium-99m ciprofloxacin (Infecton) imaging in suspected orthopaedic infection: a comparison with sequential bone/gallium imaging. Eur J Nucl Med. 28:822-30.
Yarwood J M, Bartels D J, Volper E M, Greenberg E P. (2004) Quorum sensing in Staphylococcus aureus biofilms. J Bacteriol. 186:1838-50
Yarwood J M, McCormick J K, Schlievert P M. (2001) Identification of a novel two-component regulatory system that acts in global regulation of virulence factors of Staphylococcus aureus. J Bacteriol. 183:1113-23.
Zhu M, Takenaka S, Sato M, Hoshino E. (2001) Extracellular polysaccharides do not inhibit the reaction between Streptococcus mutans and its specific immunoglobulin G. (IgG) or penetration of the IgG through S. mutans biofilm. Oral Microbiol Immunol. 16:54-6.
Zimmerli W, Trampuz A, Ochsner P E. (2004) Prosthetic-joint infections. N Engl J Med. 351:1645-54
Zlotnick A, Aldrich R, Johnson J M, Ceres P, Young M J. (2000) Mechanism of capsid assembly for an icosahedral plant virus. Virology 277:450-6.

All patents and publications referred to herein are expressly incorporated by reference in their entirety.

Although the invention has been described with reference to the presently preferred embodiments and the foregoing non-limiting examples, it should be understood that various changes and modifications, as would be obvious to one skilled in the art, can be made without departing from the spirit of the invention. Accordingly, the invention is limited only by the following claims.

What is claimed is:

1. A method of targeting a biofilm comprising contacting a biofilm with a composition comprising a protein cage.

2. A method according to claim 1, wherein said protein cage is a protein cage aggregate.

3. A method according to claim 1, wherein said protein cage penetrates said biofilm.

4. A method according to claim 1, wherein said protein cage comprises a viral protein.

5. A method according to claim 1, wherein said protein cage comprises a non-viral protein.

6. A method according to claim 1, wherein said protein cage comprises a bacterial protein.

7. A method according to claim 1, wherein said protein cage comprises at least one modified subunit.

8. A method according to claim 1, wherein said protein cage comprises at least two modified subunits.

9. A method according to claim 8, wherein said protein cage comprises more than one type of modified subunit.

10. A method according to claim 7, wherein said protein cage comprises a chemically modified subunit.

11. A method according to claim 7, wherein said protein cage comprises a genetically modified subunit.

12. A method according to claim 1, wherein said protein cage comprises one or more targeting moieties.

13. A method according to claim 1, wherein said protein cage comprises at least two targeting moieties.

14. A method according to claim 12, wherein said protein cage comprises a polylpeptide targeting moiety.

15. A method according to claim 12, wherein said protein cage comprises an antibody targeting moiety.

16. A method according to claim 1, wherein said protein cage comprises a first guest material.

17. A method according to claim 16, wherein said first guest material is a therapeutic agent.

18. A method according to claim 1, wherein said protein cage further comprises a reversible switch.

19. A method according to claim 1, wherein said protein cage is in a static open state.

20. A method according to claim 1, wherein said protein cage is in a static closed state.

21. A method according to claim 1, wherein said protein cage further comprises at least one hydrolase cleavage site.

22. A method according to claim 21, wherein said hydrolase is a protease.

23. A method according to claim 22, wherein said protease is trypsin.

24. A method according to claim 22, wherein said protease is a cathepsin.

25. A method according to claim 21, wherein said hydrolase cleavage site is located on the exterior of said protein cage.

26. A method according to claim 1, wherein said biofilm comprises Staphylococcus aureus bacteria.

27. A method according to claim 1, wherein said biofilm is adhered to tissues or biomaterials.

28. A method according to claim 1, wherein said biofilm is adhered to tissues or biomaterials in vivo.

29. A method according to claim 1, wherein said biofilm is adhered to surgical implants.

30. A method according to claim 1, wherein said biofilm is adhered to grafted biomaterial.

31. A method according to claim 1, wherein said biofilm arises from a nosocomial infection.

32. A method according to claim 1, wherein said biofilm arises from an HIV-related infection.

33. A method according to claim 1, wherein said biofilm is associated with an endocarditis-related infection.

34. A method according to claim 1, wherein said biofilm is associated with an osteomyelitis-related infection.

35. A method according to claim 1, wherein said biofilm is an antibiotic-resistant biofilm.

36. A method according to claim 17, wherein said agent penetrates said biofilm.

37. A method of imaging a cell, tissue, or biofilm comprising contacting a cell, tissue, or biofilm with a medical imaging composition comprising a protein cage.

38. A method according to claim 37, wherein said protein cage is a protein cage aggregate.

39. A method according to claim 37, wherein said medical imaging composition penetrates said cell, tissue, or biofilm.

40. A method according to claim 37, wherein said protein cage comprises a viral protein.

41. A method according to claim 37, wherein said protein cage comprises a non-viral protein.

42. A method according to claim 37, wherein said protein cage comprises a bacteria protein.

43. A method according to claim 37, wherein said protein cage comprises at least one modified subunit.

44. A method according to claim 37, wherein said protein cage comprises at least two modified subunits.

45. A method according to claim 44, wherein said protein cage comprises more than one type of modified subunit.

46. A method according to claim 43, wherein said protein cage comprises a chemically modified subunit.

47. A method according to claim 43, wherein said protein cage comprises a genetically modified subunit.

48. A method according to claim 37, wherein said protein cage comprises one or more targeting moieties.

49. A method according to claim 37, wherein said protein cage comprises at least two targeting moieties.

50. A method according to claim 48, wherein said protein cage comprises a polylpeptide targeting moiety.

51. A method according to claim 48, wherein said protein cage comprises an antibody targeting moiety.

52. A method according to claim 37, wherein said protein cage comprises a first guest material.

53. A method according to claim 37, wherein said protein cage comprises a linker.

54. A method according to claim 53, wherein said linker is a chelate.

55. A method according to claim 53, wherein said linker is a mineral phase-binding peptide.

56. A method according to claim 52, wherein said first guest material is an inorganic material.

57. A method according to claim 55, wherein said mineral phase-binding peptide further comprises an inorganic material.

58. A method according to claim 52, wherein said first guest material is a medical imaging agent.

59. A method according to claim 58, wherein said medical imaging agent is selected from the group consisting of magnetic resonance imaging (MRI) agents, nuclear magnetic resonance imaging agents (NMR), x-ray agents, optical agents, ultrasound agents and neutron capture therapy agents.

60. A method according to claim 59, wherein said imaging agent is indirectly coupled to said protein cage through said linker.

61. The method of claim 59, wherein said imaging agent is directly bound to the protein cage through chemical modification of one or more subunits.

62. A method according to claim 37, further comprising rendering an image of said cell, tissue, or biofilm.

63. A method according to claim 62, wherein said method of rendering an image is selected from the group consisting of MRI, NMR, x-ray, optical ultrasound and neutron capture therapy.

64. A method according to claim 37, wherein said protein cage comprises a mineralized inorganic material.

65. A method according to claim 64, wherein said inorganic material is a metal.

66. A method according to claim 65, wherein said metal is not iron.

67. A method according to claim 64, wherein said mineralized inorganic material is mineralized under non-physiological conditions.

68. A method according to claim 67, wherein said non-physiological conditions comprise a temperature of about 50° C. to about 70° C. and a pH of about 7.5 to about 9.

69. A method according to claim 67, wherein said non-physiological conditions comprise a temperature of about 85° C. and a pH of about 6.5.
